SEQUENCE LISTING (85 sequences)

SEQ ID NO: 1 - protein, 567 aa, synthetic construct
  source 1..567: note = wild-type PAL
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 2 - protein, 567 aa, synthetic construct
  source 1..567: note = SDPAL-01 (N25E)
MKTLSQAQSKTSSQQFSFTGNSSAEVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 3 - protein, 567 aa, synthetic construct
  source 1..567: note = SDPAL-02 (N30E)
MKTLSQAQSKTSSQQFSFTGNSSANVIIGEQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 4 - protein, 567 aa, synthetic construct
  source 1..567: note = SDPAL-03 (N44E)
MKTLSQAQSKTSSQQFSFTGNSSANVIIGNQKLTINDVARVAREGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 5 - protein, 567 aa, synthetic construct
  source 1..567: note = SDPAL-04 (T46V)
MKTLSQAQSKTSSQQFSFTGNSSANVIIGNQKLTINDVARVARNGVLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 6 - protein, 567 aa, synthetic construct
  source 1..567: note = SDPAL-05 (I56V)
MKTLSQAQSKTSSQQFSFTGNSSANVIIGNQKLTINDVARVARNGTLVSLTNNTDVLQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 7 - protein, 567 aa, synthetic construct
  source 1..567: note = SDPAL-07 (C64V)
MKTLSQAQSKTSSQQFSFTGNSSANVIIGNQKLTINDVARVARNGTLVSLTNNTDILQGIQASVDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 8 - protein, 567 aa, synthetic construct
  source 1..567: note = SDPAL-09 (Q96E)
MKTLSQAQSKTSSQQFSFTGNSSANVIIGNQKLTINDVARVARNGTLVSLTNNTDILQGIQASCDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 9 - protein, 567 aa, synthetic construct
  source 1..567: note = SDPAL-10 (L104M)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 10 - protein, 567 aa, synthetic construct
  source 1..567: note = SDPAL-11 (T110V)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKVGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 11 - protein, 567 aa, synthetic construct
  source 1..567: note = SDPAL-12 (K115Y)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNYLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 12 - protein, 567 aa, synthetic construct
  source 1..567: note = SDPAL-15 (N234L)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAALCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 13 - protein, 567 aa, synthetic construct
  source 1..567: note = SDPAL-16 (M247L)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIALGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 14 - protein, 567 aa, synthetic construct
  source 1..567: note = SDPAL-17 (D253H)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALHIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 15 - protein, 567 aa, synthetic construct
  source 1..567: note = SDPAL-19 (I285L)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 16 - protein, 567 aa, synthetic construct
  source 1..567: note = SDPAL-20 (L316I)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSIRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 17 - protein, 567 aa, synthetic construct
  source 1..567: note = SDPAL-23 (Q389N)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVNIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 18 - protein, 567 aa, synthetic construct
  source 1..567: note = SDPAL-24 (M416Q)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNQGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 19 - protein, 567 aa, synthetic construct
  source 1..567: note = SDPAL-26 (C424T)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQITGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 20 - protein, 567 aa, synthetic construct
  source 1..567: note = SDPAL-27 (N437E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGESIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 21 - protein, 567 aa, synthetic construct
  source 1..567: note = SDPAL-28 (A440S)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSISDRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 22 - protein, 567 aa, synthetic construct
  source 1..567: note = SDPAL-29 (E448D)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHADQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 23 - protein, 567 aa, synthetic construct
  source 1..567: note = SDPAL-30 (Q449E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEEFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 24 - protein, 567 aa, synthetic construct
  source 1..567: note = SDPAL-31 (Q457M)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSMGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 25 - protein, 567 aa, synthetic construct
  source 1..567: note = SDPAL-32 (S461A)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTAATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 26 - protein, 567 aa, synthetic construct
  source 1..567: note = SDPAL-34 (N474V)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQVYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 27 - protein, 567 aa, synthetic construct
  source 1..567: note = SDPAL-35 (G483A)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARACLSPATERLYSAVRHVVGQKPTSDRPYIWNDNEQGLDEHIARISADIAAGGVIVQAVQDILPCLH

SEQ ID NO: 28 - protein, 567 aa, synthetic construct
  source 1..567: note = SDPAL_37 (C503T)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARATLSPATERLYSAVRHVVGQKPTSDRPYIWNDNEQGLDEHIARISADIAAGGVIVQAVQDILPCLH

SEQ ID NO: 29 - protein, 567 aa, synthetic construct
  source 1..567: note = SDPAL-38 (Q521V)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGVKPTSDRPYIWNDNEQGLDEHIARISADIAAGGVIVQAVQDILPCLH

SEQ ID NO: 30 - protein, 567 aa, synthetic construct
  source 1..567: note = SDPAL-41 (N534D)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDDEQGLDEHIARISADIAAGGVIVQAVQDILPCLH

SEQ ID NO: 31 - protein, 568 aa, synthetic construct
  source 1..568: note = SDPAL-42 (I27V)
MKTLSQAQSKTSSQQFSFTGNSSANVVIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 32 - protein, 568 aa, synthetic construct
  source 1..568: note = SDPAL-43 (Q31R)
MKTLSQAQSKTSSQQFSFTGNSSANVIIGNRKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 33 - protein, 568 aa, synthetic construct
  source 1..568: note = SDPAL-45 (L47S)
MKTLSQAQSKTSSQQFSFTGNSSANVIIGNQKLTINDVARVARNGTSVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 34 - protein, 568 aa, synthetic construct
  source 1..568: note = SDPAL-48 (K115L)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNLLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 35 - protein, 568 aa, synthetic construct
  source 1..568: note = SDPAL-49 (L118K)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPKADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 36 - protein, 568 aa, synthetic construct
  source 1..568: note = SDPAL-50 (R134K)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMKGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 37 - protein, 568 aa, synthetic construct
  source 1..568: note = SDPAL-51 (S209A)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLAPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 38 - protein, 568 aa, synthetic construct
  source 1..568: note = SDPAL-54 (A246S)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAISMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 39 - protein, 568 aa, synthetic construct
  source 1..568: note = SDPAL-55 (D282A)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAAAQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 40 - protein, 568 aa, synthetic construct
  source 1..568: note = SDPAL-56 (I310V)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELVQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 41 - protein, 568 aa, synthetic construct
  source 1..568: note = SDPAL-58 (R313K)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDKYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 42 - protein, 568 aa, synthetic construct
  source 1..568: note = RDPAL-02 (Q96E, G483A, C503T)
MKTLSQAQSKTSSQQFSFTGNSSANVIIGNQKLTINDVARVARNGTLVSLTNNTDILQGIQASCDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLHG

SEQ ID NO: 43 - protein, 568 aa, synthetic construct
  source 1..568: note = RDPAL-16 (I56V, Q457M, Q521V)
MKTLSQAQSKTSSQQFSFTGNSSANVIIGNQKLTINDVARVARNGTLVSLTNNTDVLQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSMGYTSATLARRSVDIFQNYVAIALMFGVQAVDLRTYKKTGHYDARACLSPATERLYSAVRHVVGVKPTSDRPYIWNDNEQGLDEHIARISADIAAGGVIVQAVQDILPCLHG

SEQ ID NO: 44 - protein, 568 aa, synthetic construct
  source 1..568: note = RDPAL-17 (I56V, S461A, Q521V)
MKTLSQAQSKTSSQQFSFTGNSSANVIIGNQKLTINDVARVARNGTLVSLTNNTDVLQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTAATLARRSVDIFQNYVAIALMFGVQAVDLRTYKKTGHYDARACLSPATERLYSAVRHVVGVKPTSDRPYIWNDNEQGLDEHIARISADIAAGGVIVQAVQDILPCLHG

SEQ ID NO: 45 - protein, 568 aa, synthetic construct
  source 1..568: note = RDPAL-20 (N30E, Q96E, I285L)
MKTLSQAQSKTSSQQFSFTGNSSANVIIGEQKLTINDVARVARNGTLVSLTNNTDILQGIQASCDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 46 - protein, 568 aa, synthetic construct
  source 1..568: note = RDPAL-21 (N30E, Q96E, C503T)
MKTLSQAQSKTSSQQFSFTGNSSANVIIGEQKLTINDVARVARNGTLVSLTNNTDILQGIQASCDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARATLSPATERLYSAVRHVVGQKPTSDRPYIWNDNEQGLDEHIARISADIAAGGVIVQAVQDILPCLHG

SEQ ID NO: 47 - protein, 568 aa, synthetic construct
  source 1..568: note = RDPAL-22 (C64V, L104M, C503T)
MKTLSQAQSKTSSQQFSFTGNSSANVIIGNQKLTINDVARVARNGTLVSLTNNTDILQGIQASVDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARATLSPATERLYSAVRHVVGQKPTSDRPYIWNDNEQGLDEHIARISADIAAGGVIVQAVQDILPCLHG

SEQ ID NO: 48 - protein, 568 aa, synthetic construct
  source 1..568: note = RDPAL-18 (N25E, C64V, M247L, Q457M)
MKTLSQAQSKTSSQQFSFTGNSSAEVIIGNQKLTINDVARVARNGTLVSLTNNTDILQGIQASVDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIALGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSMGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 49 - protein, 568 aa, synthetic construct
  source 1..568: note = RDPAL-19 (N25E, C64V, M247L, S461A)
MKTLSQAQSKTSSQQFSFTGNSSAEVIIGNQKLTINDVARVARNGTLVSLTNNTDILQGIQASVDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIALGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTAATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 50 - protein, 568 aa, synthetic construct
  source 1..568: note = RDPAL-23 (N30E, Q96E, I285L, C503T)
MKTLSQAQSKTSSQQFSFTGNSSANVIIGEQKLTINDVARVARNGTLVSLTNNTDILQGIQASCDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARATLSPATERLYSAVRHVVGQKPTSDRPYIWNDNEQGLDEHIARISADIAAGGVIVQAVQDILPCLHG

SEQ ID NO: 51 - protein, 568 aa, synthetic construct
  source 1..568: note = RDPAL-24 (C64V, L104M, I285L, Q521V)
MKTLSQAQSKTSSQQFSFTGNSSANVIIGNQKLTINDVARVARNGTLVSLTNNTDILQGIQASVDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGVKPTSDRPYIWNDNEQGLDEHIARISADIAAGGVIVQAVQDILPCLHG

SEQ ID NO: 52 - protein, 568 aa, synthetic construct
  source 1..568: note = RPDAL-25 (N30E, L104M, I285L, Q521V)
MKTLSQAQSKTSSQQFSFTGNSSANVIIGEQKLTINDVARVARNGTLVSLTNNTDILQGIQASCDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGVKPTSDRPYIWNDNEQGLDEHIARISADIAAGGVIVQAVQDILPCLHG

SEQ ID NO: 53 - protein, 568 aa, synthetic construct
  source 1..568: note = RDPAL-05 (A70S, Q96E, G483A, C503T, N30E)
MKTLSQAQSKTSSQQFSFTGNSSANVIIGEQKLTINDVARVARNGTLVSLTNNTDILQGIQASCDYINNSVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLHG

SEQ ID NO: 54 - protein, 568 aa, synthetic construct
  source 1..568: note = RDPAL-06 (A70S, Q96E, G483A, C503T, L104M)
MKTLSQAQSKTSSQQFSFTGNSSANVIIGNQKLTINDVARVARNGTLVSLTNNTDILQGIQASCDYINNSVESGEPIYGVTSGFGGMANVAISREEASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLHG

SEQ ID NO: 55 - protein, 568 aa, synthetic construct
  source 1..568: note = "RDPAL-11 (L104M, C64V, N30E, G483A, C503T) "
MKTLSQAQSKTSSQQFSFTGNSSANVIIGEQKLTINDVARVARNGTLVSLTNNTDILQGIQASVDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLHG

SEQ ID NO: 56 - protein, 568 aa, synthetic construct
  source 1..568: note = RDPAL-12 (Q96E, C64V, N30E, G483A, C503T)
MKTLSQAQSKTSSQQFSFTGNSSANVIIGEQKLTINDVARVARNGTLVSLTNNTDILQGIQASVDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLHG

SEQ ID NO: 57 - protein, 568 aa, synthetic construct
  source 1..568: note = RDPAL-13 (L104M, C64V, N30E, G483A, Q521V)
MKTLSQAQSKTSSQQFSFTGNSSANVIIGEQKLTINDVARVARNGTLVSLTNNTDILQGIQASVDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARACLSPATERLYSAVRHVVGVKPTSDRPYIWNDNEQGLDEHIARISADIAAGGVIVQAVQDILPCLHG

SEQ ID NO: 58 - protein, 568 aa, synthetic construct
  source 1..568: note = RDPAL-14 (Q96E, C64V, N30E, G483A, Q521V)
MKTLSQAQSKTSSQQFSFTGNSSANVIIGEQKLTINDVARVARNGTLVSLTNNTDILQGIQASVDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARACLSPATERLYSAVRHVVGVKPTSDRPYIWNDNEQGLDEHIARISADIAAGGVIVQAVQDILPCLHG

SEQ ID NO: 59 - protein, 568 aa, synthetic construct
  source 1..568: note = RDPAL-07 (A70S, Q96E, G483A, C503T, I285L, Q521V)
MKTLSQAQSKTSSQQFSFTGNSSANVIIGNQKLTINDVARVARNGTLVSLTNNTDILQGIQASCDYINNSVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMLSLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGVKPTSDRPYIWNDNEQGLDEHIARISADIAAGGVIVQAVQDILPCLHG

SEQ ID NO: 60 - protein, 568 aa, synthetic construct
  source 1..568: note = RDPAL-09 (Q96E, C64V, N30E, G483A, C503T, Q521V)
MKTLSQAQSKTSSQQFSFTGNSSANVIIGEQKLTINDVARVARNGTLVSLTNNTDILQGIQASVDYINNAVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGVKPTSDRPYIWNDNEQGLDEHIARISADIAAGGVIVQAVQDILPCLHG

SEQ ID NO: 61 - protein, 568 aa, synthetic construct
  source 1..568: note = RDPAL-10 (L104M, C64V, N30E, G483A, C503T, Q521V)
MKTLSQAQSKTSSQQFSFTGNSSANVIIGEQKLTINDVARVARNGTLVSLTNNTDILQGIQASVDYINNAVESGEPIYGVTSGFGGMANVAISREQASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGVKPTSDRPYIWNDNEQGLDEHIARISADIAAGGVIVQAVQDILPCLHG

SEQ ID NO: 62 - protein, 568 aa, synthetic construct
  source 1..568: note = RDPAL-08 (N30E, C64V, A70S, Q96E, L104M, G483A, C503T, Q521V)
MKTLSQAQSKTSSQQFSFTGNSSANVIIGEQKLTINDVARVARNGTLVSLTNNTDILQGIQASVDYINNSVESGEPIYGVTSGFGGMANVAISREEASELQTNMVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGVKPTSDRPYIWNDNEQGLDEHIARISADIAAGGVIVQAVQDILPCLHG

SEQ ID NO: 63 - protein, 567 aa, synthetic construct
  source 1..567: note = SDPAL-06 (I56T)
MKTLSQAQSKTSSQQFSFTGNSSANVIIGNQKLTINDVARVARNGTLVSLTNNTDTLQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 64 - protein, 566 aa, synthetic construct
  source 1..566: note = SPDAL-08 (A70S)
MKTLSQAQSKTSSQQFSFTGNSSANVIIGNQKLTINDVARVARNGTLVSLTNNTDILQGIQASCDYINNSVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNREKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 65 - protein, 567 aa, synthetic construct
  source 1..567: note = SDPAL-13 (Y160K)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVKEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 66 - protein, 567 aa, synthetic construct
  source 1..567: note = SDPAL-14 (F188Y)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSYKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 67 - protein, 567 aa, synthetic construct
  source 1..567: note = SDPAL-18 (D253F)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALFIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 68 - protein, 567 aa, synthetic construct
  source 1..567: note = SDPAL-21 (N347D)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDDPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 69 - protein, 567 aa, synthetic construct
  source 1..567: note = SDPAL-22 (Q366M)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGMYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 70 - protein, 567 aa, synthetic construct; identical to SEQ ID NO: 17
  source 1..567: note = SDPAL-25 (Q422E)

SEQ ID NO: 71 - protein, 567 aa, synthetic construct
  source 1..567: note = SDPAL-33 (Q473K)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFKNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 72 - protein, 567 aa, synthetic construct
  source 1..567: note = SDPAL-36 (G483S)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFSVQAVDLRTYKKTGHYDARACLSPATERLYSAVRHVVGQKPTSDRPYIWNDNEQGLDEHIARISADIAAGGVIVQAVQDILPCLH

SEQ ID NO: 73 - protein, 567 aa, synthetic construct
  source 1..567: note = SDPAL-39 (Q521E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGEKPTSDRPYIWNDNEQGLDEHIARISADIAAGGVIVQAVQDILPCLH

SEQ ID NO: 74 - protein, 567 aa, synthetic construct
  source 1..567: note = SDPAL-40 (Y529F)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPFIWNDNEQGLDEHIARISADIAAGGVIVQAVQDILPCLH

SEQ ID NO: 75 - protein, 568 aa, synthetic construct
  source 1..568: note = SDPAL-44 (R43Y)
MKTLSQAQSKTSSQQFSFTGNSSANVIIGNQKLTINDVARVAYNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 76 - protein, 568 aa, synthetic construct
  source 1..568: note = SDPAL-46 (E72K)
MKTLSQAQSKTSSQQFSFTGNSSANVIIGNQKLTINDVARVARNGTLVSLTNNTDILQGIQASCDYINNAVKSGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 77 - protein, 568 aa, synthetic construct
  source 1..568: note = SDPAL-47 (I92D)
MKTLSQAQSKTSSQQFSFTGNSSANVIIGNQKLTINDVARVARNGTLVSLTNNTDILQGIQASCDYINNAVESGEPIYGVTSGFGGMANVADSREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 78 - protein, 568 aa, synthetic construct
  source 1..568: note = SDPAL-52 (K216R)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPREGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 79 - protein, 568 aa, synthetic construct
  source 1..568: note = SDPAL-53 (N223E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMEGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 80 - protein, 568 aa, synthetic construct
  source 1..568: note = SDPAL-57 (Q311E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIEDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 81 - protein, 568 aa, synthetic construct
  source 1..568: note = SDPAL-59 (Q321E)
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPEYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHG

SEQ ID NO: 82 - protein, 567 aa, synthetic construct
  source 1..567: note = RDPAL-01 (A70S, Q96E, G483A)
MKTLSQAQSKTSSQQFSFTGNSSANVIIGNQKLTINDVARVARNGTLVSLTNNTDILQGIQASCDYINNSVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARACLSPATERLYSAVRHVVGQKPTSDRPYIWNDNEQGLDEHIARISADIAAGGVIVQAVQDILPCLH

SEQ ID NO: 83 - protein, 567 aa, synthetic construct
  source 1..567: note = RDPAL-03 (A70S, G483A, C503T)
MKTLSQAQSKTSSQQFSFTGNSSANVIIGNQKLTINDVARVARNGTLVSLTNNTDILQGIQASCDYINNSVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLH

SEQ ID NO: 84 - protein, 567 aa, synthetic construct
  source 1..567: note = RDPAL-04 (A70S, Q96E, G483A, C503T)
MKTLSQAQSKTSSQQFSFTGNSSANVIIGNQKLTINDVARVARNGTLVSLTNNTDILQGIQASCDYINNSVESGEPIYGVTSGFGGMANVAISREEASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFAVQAVDLRTYKKTGHYDARATLSPATERLYSAVRHVVGQKPTSDRPYIWNDNEQGLDEHIARISADIAAGGVIVQAVQDILPCLH

SEQ ID NO: 85 - protein, 567 aa, synthetic construct
  source 1..567: note = RDPAL-15 (I56V, M247L, Q457M)
MKTLSQAQSKTSSQQFSFTGNSSANVIIGNQKLTINDVARVARNGTLVSLTNNTDVLQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIALGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSMGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

(19) United States
(12) Patent Application Publication
(10) Pub. No.: US 2025/0295742 A1
(43) Pub. Date: Sep. 25, 2025

(54) PHENYLALANINE-DEGRADING ENZYME VARIANTS AND TREATMENTS FOR PHENYLKETONURIA

(71) Applicant: Iditarod Bio, Inc., Raleigh, NC (US)

(72) Inventors: Austin Woodrow Cole, Raleigh, NC (US); Manish Sharma, Waterloo (CA); Som Dutt, Dehradoon (IN)

(73) Assignee: Iditarod Bio, Inc., Raleigh, NC (US)

(21) Appl. No.: 19/060,328

(22) Filed: Feb. 21, 2025

Related U.S. Application Data
(60) Provisional application No. 63/556,557, filed on Feb. 22, 2024.

(51) Int. Cl. A61K 38/51; C12N 9/88

(52) U.S. Cl. CPC A61K 38/51; C12N 9/88; C12Y 403/01024

(57) ABSTRACT

Provided herein are phenylalanine-degrading enzyme variants for use in degrading phenylalanine and in treating phenylketonuria (PKU). More specifically, provided herein are phenylalanine-degrading enzyme variants that exhibit increased thermal stability and physicochemical resistance as measured by residual phenylalanine-degrading activity following challenge.

### CROSS-REFERENCE TO RELATED APPLICATION

This application claims the benefit under 35 U.S.C. § 119(e) of U.S. Provisional Patent Application No. 63/556,557, filed Feb. 22, 2024, the entire contents of which is incorporated herein by reference in its entirety.

### SEQUENCE LISTING

The instant application contains a Sequence Listing which is hereby incorporated by reference in its entirety. Said Sequence Listing xml file was created on May 5, 2025, is 13 kilobytes in size, and is named 123818_0401_Sequence_Listing.xml.

### TECHNICAL FIELD

This disclosure relates to enzyme variants for use in treating disease. More particularly, this disclosure relates to phenylalanine-degrading enzyme variants that may be used in the treatment of phenylketonuria, along with pharmaceutical compositions and methods of treatment using said variants.

### BACKGROUND

The following description of the background of the present technology is provided simply as an aid in understanding the present technology and is not admitted to describe or constitute prior art to the present technology.

Phenylketonuria (PKU) is a rare metabolic disorder typically caused by a mutation in the phenylalanine hydroxylase (PAH) gene. PAH is responsible for breaking down the amino acid phenylalanine that is ingested, and loss of function of the enzyme results in build-up of phenylalanine in the body, resulting in serious health problems including brain damage.

The incidence of PKU varies in different countries, with the annual incidence rate in the United States being between 1 in 10,000 to 1 in 15,000 live births. As PKU can be managed if detected early, babies born in the U.S. and other countries are screened for the disorder soon after birth. Typical treatments for PKU focus on restricting the diet of the subject to limit the amount of phenylalanine that is consumed; however, use of only a dietary-based treatment process still increases the incidence of attention deficit hyperactivity disorder and other specific learning disabilities in patients with the disorder. Thus, development of alternative treatment approaches beyond simple dietary restriction methods is desirable.

### SUMMARY

The present disclosure relates to phenylalanine-degrading enzyme variants, compositions of said enzyme variants and methods of use of said variants. As shown herein, administration of the enzyme variant can improve phenylalanine degradation capabilities in a subject that may have phenylketonuria. The present disclosure shows that the enzyme variants may exhibit increased thermal stability and increased residual activity following application of physicochemical stress. It is to be understood that the disclosed embodiments are merely exemplary, and accordingly, the invention may be embodied in various and alternative forms. The specific structural and functional details disclosed herein are not to be interpreted as limiting but merely as a basis for the claims and as a representative basis for teaching one skilled in the art to variously employ the embodiments described herein.

In one aspect, the present disclosure provides phenylalanine-degrading enzyme variants comprising an amino acid sequence that is a variant of the amino acid sequence of the wild-type phenylalanine ammonia lyase (PAL) enzyme set forth in SEQ ID NO: 1, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1.

In some embodiments, the substitution increases the thermostability of the variant relative to the wild-type PAL enzyme. In some embodiments, the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C. In some embodiments, the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C. In some embodiments, the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

In some embodiments, the substitution increases the residual PAL activity following nebulization relative to the wild-type PAL enzyme. In some embodiments, the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge. In some embodiments, the amount of PAL activity is determined by measuring the amount of cinnamic acid produced via liquid chromatography with UV detection following a 30-minute incubation of 21 μg of nebulization challenged enzyme with 25 μL of 80 mM phenylalanine. In some embodiments, the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

In some embodiments, the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

In some embodiments, the variant sequence comprises an amino acid sequence selected from any one of SEQ ID NOs: 2-41. In some embodiments, the variant sequence comprises at least two substitutions at two or more amino acid positions selected from 25, 30, 56, 64, 70, 96, 104, 247, 285, 457, 461, 483, 503, and 521 of SEQ ID NO: 1.

In some embodiments, the variant sequence comprises at least three substitutions at three or more amino acid positions selected from 25, 30, 56, 64, 70, 96, 104, 247, 285, 457, 461, 483, 503, and 521 of SEQ ID NO: 1. In some embodiments, the variant sequence comprises at least three substitutions selected from N25E, N30E, I56V, C64V, A70S, Q96E, L104M, M247L, I285L, Q457M, S461A, G483A, C503T, and Q521V. In some embodiments, the variant sequence comprises an amino acid sequence selected from any one of SEQ ID NOs: 42-47.

In some embodiments, the variant sequence comprises at least four substitutions at four or more amino acid positions selected from 25, 30, 64, 70, 96, 104, 247, 285, 457, 461, 483, 503, and 521 of SEQ ID NO: 1. In some embodiments, the variant sequence comprises at least four substitutions selected from N25E, N30E, C64V, A70S, Q96E, L104M, M247L, I285L, Q457M, S461A, G483A, C503T, and Q521V. In some embodiments, the variant sequence comprises an amino acid sequence selected from any one of SEQ ID NOs: 48-52.

In some embodiments, the variant sequence comprises at least five substitutions at five or more amino acid positions selected from 30, 64, 70, 96, 104, 483, 503, and 521 of SEQ ID NO: 1. In some embodiments, the variant sequence comprises at least five substitutions selected from N30E, C64V, A70S, Q96E, L104M, G483A, C503T, and Q521V. In some embodiments, the variant sequence comprises an amino acid sequence selected from any one of SEQ ID NOs: 53-58.

In some embodiments, the variant sequence comprises at least six substitutions at six or more amino acid positions selected from 30, 64, 70, 96, 104, 483, 503, and 521 of SEQ ID NO: 1. In some embodiments, the variant sequence comprises at least six substitutions selected from N30E, C64V, A70S, Q96E, L104M, G483A, C503T, and Q521V. In some embodiments, the variant sequence comprises an amino acid sequence selected from any one of SEQ ID NOs: 59-61.

In some embodiments, the variant sequence comprises at least eight substitutions at eight or more amino acid positions selected from 30, 64, 70, 96, 104, 483, 503, and 521 of SEQ ID NO: 1. In some embodiments, the variant sequence comprises at least eight substitutions selected from N30E, C64V, A70S, Q96E, L104M, G483A, C503T, and Q521V. In some embodiments, the variant sequence comprises an amino acid sequence of SEQ ID NO: 62.

In some embodiments, the variant sequence comprises a deletion of a methionine residue at amino acid position 1 of SEQ ID NO: 1.

In another aspect, the present disclosure provides enzyme variants for use in treating phenylketonuria (PKU) (e.g., an enzyme variant of any one of the forgoing aspect or embodiments).

In another aspect, the present disclosure provides uses of an enzyme variant disclosed herein (e.g., an enzyme variant of any one of the forgoing aspects or embodiments) in the manufacture of a medicament for treating phenylketonuria (PKU).

In another aspect, the present disclosure provides pharmaceutical compositions, comprising: (a) a phenylalanine-degrading enzyme variant disclosed herein (e.g., an enzyme variant of any one of the forgoing aspects or embodiments), and (b) a pharmaceutically acceptable carrier or excipient. In some embodiments, the pharmaceutical composition is formulated for aerosol administration. In some embodiments, the aerosol administration is selected from liquid aerosol administration or dry powder aerosol administration.

In another aspect, the present disclosure provides pharmaceutical compositions, comprising: a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme; and a pharmaceutically acceptable carrier.

In some embodiments, the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C. In some embodiments, the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C. In some embodiments, the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

In some embodiments, the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge. In some embodiments, the amount of PAL activity is determined by measuring the amount of cinnamic acid produced via liquid chromatography with UV detection following a 30-minute incubation of 21 μg of nebulization challenged enzyme with 25 μL of 80 mM phenylalanine. In some embodiments, the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

In some embodiments, the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

In some embodiments, the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, I285L, I310V, R313K, L316I, Q389N, M416Q, C424T, N437E, A440S, E448D, Q449E, Q457M, S461A, N474V, G483A, C503T, Q521V, and N534D.

In some embodiments, the variant sequence comprises an amino acid sequence selected from any one of SEQ ID NOs: 2-62.

In some embodiments, the pharmaceutical composition is formulated for aerosol administration. In some embodiments, the aerosol administration is selected from liquid aerosol administration or solid (e.g., dry powder) aerosol administration. In another aspect, the present disclosure provides the disclosed pharmaceutical compositions (e.g., any of the foregoing aspect of embodiments) for use in treating phenylketonuria (PKU).

In another aspect, the present disclosure provides uses of the disclosed pharmaceutical compositions (e.g., any of the foregoing aspect of embodiments) in the manufacture of a medicament for treating phenylketonuria (PKU).

In another aspect, the present disclosure provides methods of treating a subject with PKU, the method comprising administering to the subject a variant disclosed herein or a pharmaceutical composition disclosed here (e.g., any of the foregoing aspects or embodiments). In some embodiments, the variant or the pharmaceutical composition is administered as a liquid aerosol. In some embodiments, the variant or the pharmaceutical composition is administered as a solid (e.g., dry powder) aerosol.

In another aspect, the present disclosure provides methods of treating a subject with PKU, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.

In some embodiments, the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C. In some embodiments, the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C. In some embodiments, the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

In some embodiments, the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge. In some embodiments, the amount of PAL activity is determined by measuring the amount of cinnamic acid produced via liquid chromatography with UV detection following a 30-minute incubation of, on average, 21 μg of nebulization challenged enzyme with 25 μL of 80 mM phenylalanine. In some embodiments, the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

In some embodiments, the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

In some embodiments, the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, I285L, I310V, R313K, L316I, Q389N, M416Q, C424T, N437E, A440S, E448D, Q449E, Q457M, S461A, N474V, G483A, C503T, Q521V, and N534D.

In some embodiments, the variant sequence comprises two to fourteen substitutions at two to fourteen amino acid positions selected from 25, 30, 56, 64, 70, 96, 104, 247, 285, 457, 461, 483, 503, and 521 of SEQ ID NO: 1. In some embodiments, the two to fourteen substitutions are selected from N25E, N30E, I56V, C64V, A70S, Q96E, L104M, M247L, I285L, Q457M, S461A, G483A, C503T, and Q521V. In some embodiments, the variant sequence comprises 2, 3, 4, 5, 6, 7, 8, 9, 10, 11, 12, 13, 14 or more substitutions.

In some embodiments, the variant sequence comprises an amino acid sequence selected from SEQ ID NO: 2-62.

In some embodiments, the enzyme variant is formulated for aerosol administration. In some embodiments, the aerosol administration is selected from liquid aerosol administration or dry powder aerosol administration.

In another aspect, the present disclosure provides methods of degrading phenylalanine in a subject, the method comprising administering to a variant disclosed herein or a pharmaceutical composition disclosed here (e.g., any of the foregoing aspects or embodiments). In some embodiments, the variant or the pharmaceutical composition is administered as a liquid aerosol. In some embodiments, the variant or the pharmaceutical composition is administered as a dry powder aerosol. In some embodiments, the subject has PKU.

In another aspect, the present disclosure provides methods of degrading phenylalanine in a subject, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.

In some embodiments, the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C. In some embodiments, the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C. In some embodiments, the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

In some embodiments, the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge. In some embodiments, the amount of PAL activity is determined by measuring the amount of cinnamic acid produced via liquid chromatography with UV detection following a 30-minute incubation of 21 μg of nebulization challenged enzyme with 25 μL of 80 mM phenylalanine. In some embodiments, the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

In some embodiments, the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

In some embodiments, the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

In some embodiments, the variant sequence comprises two to fourteen substitutions at two to fourteen amino acid positions selected from 25, 30, 56, 64, 70, 96, 104, 247, 285, 457, 461, 483, 503, and 521 of SEQ ID NO: 1. In some embodiments, the two to fourteen substitutions are selected from N25E, N30E, I56V, C64V, A70S, Q96E, L104M, M247L, I285L, Q457M, S461A, G483A, C503T, and Q521V. In some embodiments, the variant sequence comprises 2, 3, 4, 5, 6, 7, 8, 9, 10, 11, 12, 13, or 14 substitutions.

In some embodiments, the variant sequence comprises an amino acid sequence selected from SEQ ID NO: 2-62.

In some embodiments, the enzyme variant is formulated for aerosol administration. In some embodiments, the aerosol administration is selected from liquid aerosol administration or dry powder aerosol administration. In some embodiments, the subject has phenylketonuria (PKU).

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

FIG. 1 depicts the activity of various wild-type PAL enzymes as measured by the amount of cinnamic produced with three different enzyme loads for each enzyme. SPDAL_00_T1 and SDPAL_00_T2 are both generated Anabaena variabilis PAL enzymes. PAL Merck is the Rhodotorula glutinis recombinant PAL enzyme that is commercially available.

FIGS. 2A and 2B depict wild-type PAL enzymatic activity as measured by the amount of cinnamic acid released at various temperatures. FIG. 2A shows the activity of the wild-type PAL enzyme at 30° C. and at ten-degree increments from 50° C. to 90° C. FIG. 2B shows the activity of wild-type PAL enzyme at one- to two-degree increments from 70° C. to 78° C.

FIGS. 3A, 3B, and 3C depict the activity of the PAL variants generated in the first round of variant creation as a measure of released cinnamic acid. PAL activity was measured as the amount of cinnamic acid released due to PAL degradation of phenylalanine. FIG. 3A shows PAL activity for the variants without thermal challenge. FIG. 3B shows PAL activity for the variants following thermal challenge. FIG. 3C illustrates the normalized activity before thermal challenge on the X-axis for each enzyme variant, the normalized activity following thermal challenge on the Y-axis for each enzyme variant, and the concentration of the variant produced by the third party provided Tierra.

FIG. 4 depicts the activity of the PAL variants generated in the second round of variant creation as a measure of released cinnamic acid. Each enzyme has four data points. The blue and red bars are replicates of enzyme activity determined following thermal challenged. The green and purple bars are replicates of enzyme activity determined without thermal challenge.

FIG. 5 shows the typical SDS-PAGE gel of PAL variants and wild-type enzyme purified from shake flask culture of enzyme-expressing Escherichia coli. The shake flask protocol utilized 100 mL of medium in a 500 mL flask. SF1 corresponds to RDPAL_9, SF2 corresponds to RDPAL_24, SF3 corresponds to RDPAL_20, SF4 corresponds to RDPAL_11, and SF5 corresponds to wild-type PAL. The first two lanes for each enzyme represent the supernatant fraction. The second two lanes for each enzyme represent the pellet fraction.

FIG. 6 shows the exemplary SDS-PAGE gel of the PAL variant RDPAL_9 at the various stages of purification.

FIG. 7 depicts the SDS-PAGE gel of E. coli-made PAL variants and wild-type PAL enzyme before and after nebulization.

### DETAILED DESCRIPTION OF THE DRAWINGS

Phenylketonuria, also called PKU, is a rare inherited disorder that causes an amino acid called phenylalanine to build up in the body. PKU is caused by a change in the phenylalanine hydroxylase (PAH) gene. This gene helps create the enzyme needed to break down phenylalanine. Currently, there is no cure for PKU and treatment options are limited.

In addition to dietary-based treatments, a handful of protein therapy treatment options have been developed. One such therapy is a daily subcutaneous injection of a purified and PEGylated recombinant phenylalanine ammonia lyase (PAL) enzyme from A. variabilis. This treatment option has only been approved by the FDA for use in adults and is known to cause severe allergic reactions. In addition, the treatment administration caused some subjects to be less compliant than for other treatment options. Other orally administered medications have been studied, some of which also involve administration of the PAL enzymes to subjects, but currently, none of these treatments have been approved for use.

Provided herein are phenylalanine-degrading enzyme variants that may be used to treat PKU, along with a pharmaceutical composition and treatments utilizing the same. The disclosed variants modified forms of the wild-type PAL enzyme, wherein the variant exhibits increased thermostability relative to the wild-type PAL enzyme and increased residual PAL activity following nebulization relative to the wild-type PAL enzyme. The variants can be delivered to a subject through a liquid aerosol administration or a dry powder aerosol administration. The variants with increased thermostability and residual PAL activity following nebulization are suited for this administration method, these methods subject the enzyme variants to physical stressors that are not presented in other methods of administration. Such a method of treatment may facilitate increased compliance with the treatment regiment as it is not invasive, painful, or unduly limiting for the patient. However, enzyme variants with increased thermostability and residual PAL activity may be administered via other methods of administration in addition to aerosols (e.g., oral administration, injection, etc.).

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein for the purpose of describing particular embodiments only and is not intended to be limiting.

### I. Definitions

Unless defined otherwise, all technical and scientific terms used herein generally have the same meaning as commonly understood by one of ordinary skill in the art to which this technology belongs. As used in this specification and the appended claims, the singular forms “a,” “an,” and “the” include plural referents unless the context clearly dictates otherwise. For example, reference to a “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value). Additionally, when “about” is stated with respect to a particular value, it should be understood as the stated number and up to 10% plus or minus that number. For example, the phrase “about 10” should be understood as disclosed both 10 and a range of 9-11.

As used herein, the phrases “therapeutically effective amount” and “therapeutic level” mean a PAL enzyme variant dosage or plasma concentration in a subject that provides the specific pharmacological effect for which the PAL enzyme variant is administered to a subject in need of such treatment, i.e. to facilitate degradation of phenylalanine and to mitigate symptoms of PKU. It is emphasized that a therapeutically effective amount or therapeutic level of the PAL enzyme variant will not always be effective in treating the PKU symptoms of a given subject, even though such dosage is deemed to be a therapeutically effective amount by those of skill in the art. For convenience only, exemplary amounts are provided below.

Those skilled in the art can adjust such amounts in accordance with standard practices as needed to treat a specific subject. The therapeutically effective amount may vary based on the route of administration and dosage form, the age and weight of the subject and/or the subject's condition, including the amount of phenylalanine in the subject's bloodstream at the time of treatment, the amount of phenylalanine generally consumed/ingested by the subject, and/or the degree of PKU disease manifestation in the subject.

The terms of “treatment” or “treating” as sued herein with reference to PKU refer to one or more of: reducing, ameliorating or eliminating one or more symptoms or effects of phenylalanine accumulation; reducing the subject's blood levels of phenylalanine; and/or reducing the amount of phenylalanine localized in specific tissues of the subject.

The terms “individual,” “subject,” and “patient” are used interchangeably herein, and refer to any individual mammal subject, e.g., bovine, canine, feline, equine, or human.

### II. Phenylketonuria

Phenylketonuria (PKU) is an aminoacidopathy or a disease wherein the accumulation of an amino acid or its metabolite is toxic. The disease is caused by a missense mutation in a gene for an enzyme or cofactor used to degrade the amino acid phenylalanine. The traditional or classic form of PKU is caused by mutations in the q22-24 region of chromosome 12, which results in defects in the structure and function in phenylalanine hydroxylase (PAH). Less severe forms of PKU may be caused by other mutations in PAH that may affect binding of the necessary cofactor tetrahydrobiopterin (BH4) or by mutations in the BH4 gene. When PAH activity is decreased or eliminated, phenylalanine accumulates in the subject's blood and tissues, including the central nervous system. The disease is characterized by elevated levels of phenylalanine and decreased levels of tyrosine in the blood.

PKU is a progressive disease that generally does not cause acute symptoms. A child subject with PKU is completely asymptomatic, and the disease can only be detected via a blood test. Untreated PKU causes progressive brain damage that affects cognitive function while not affecting life-sustaining structures in the brain such that affected patients typically reach adulthood. Affected individuals may exhibit motor defects.

Treatment usually consists of a diet that limits protein intake to avoid phenylalanine intake. Subjects following restrictive diet treatment must have their diet supplemented with special protein and vitamin products that lack phenylalanine as the dietary regimen is inherently nutritionally deficient. While the diet-based therapy ameliorates the major neurological defects associated with the disease, individuals using the therapy do have a higher incidence of anxiety, depression, and executive function deficits.

A minority of patients can be treated with the PAH cofactor, BH4, which enhances PAH residual activity, facilitating the reduction of circulating phenylalanine. An enzyme-based therapy for PKU currently exists. It utilizes a purified and PEGylated form of recombinant PAL derived from Anabaena variabilis. The therapy has been approved by the U.S. Food and Drug Administration as a prescription treatment for PKU only for adults. The therapy is administered via daily subcutaneous injections. This treatment option poses certain difficulties for effective management of PKU. First, it is only available for adults; therefore, children still must rely on phenylalanine-restricted diets, which may give rise to the same executive function and emotional disorders for subjects following the diet-based therapy. Second, the enzyme treatment is known to cause severe allergic reactions in some subjects. Finally, the injection administration approach may make it more difficult for patients using this therapy option to adhere to their treatment plan. Thus, development of therapies that have fewer negative side effects, can be administered to more patient populations, and allow for increased adherence to treatment plans is still a pressing need.

### III. Phenylalanine Degradation Enzymes

There are two classes of enzymes known to degrade phenylalanine. The first is phenylalanine hydroxylase (PAH). PAH is an enzyme that catalyzes the hydroxylation of the aromatic sidechain of phenylalanine to generate tyrosine. PAH is one of three members of the biopterin-dependent aromatic amino acid hydroxylases, a class of monooxygenases that uses tetrahydrobiopterin (BH₄), which is a pteridine cofactor, and a non-heme iron for catalysts.

The second is phenylalanine ammonia lyase (PAL). PAL catalyzes the conversion of L-phenylalanine to ammonia and trans-cinnamic acid. Crystallographic studies have revealed that PAL is mostly a homotetrameric protein. PAL does not require the activity of other cofactors or catalysts to degrade phenylalanine.

Use of PAL to treat PKU may present advantages over use of PAH. Some forms of PKU may be a result of mutations in the gene encoding BH4; this form of PKU would not benefit from administration of additional PAH as PAH requires BH4 to degrade phenylalanine. In addition, PAL does not require other cofactors to facilitate degradation and thus may act to degrade phenylalanine alone, making it a more viable treatment candidate.

### IV. PAL Enzyme Variants

Disclosed herein are variants of a phenylalanine-degrading enzyme for use in treating PKU and in degrading phenylalanine in the bloodstream of a subject. The variants may be selected based on their thermostability and residual enzyme activity following physicochemical stress, including nebulization. The phenylalanine-degrading enzyme variants may be variants of the wild-type PAL enzyme. The phenylalanine-degrading enzyme variants may be variants of the wild-type PAH enzyme.

At least one mutation that increases the thermostability of the PAL enzyme may be introduced into the variant, allowing the variant to remain active and degrade phenylalanine at higher temperatures or following thermal challenge as compared to the wild-type PAL enzyme. The increase in thermostability may be measured by comparing the amount of cinnamic acid, a phenylalanine degradation product, produced by the enzyme variant and the wild-type PAL enzyme following thermal challenge. The thermal challenge may be a 10-minute incubation period at 74° C. The amount of PAL activity may be detected by measuring the amount of cinnamic acid measured via liquid chromatography with UV detection following a 30-minute incubation period of 25 μL of 0.7 to 1 μg/μL unchallenged and thermal challenged enzyme with 25 μL of 80 mM phenylalanine. A variant may be selected for use to degrade phenylalanine or to treat PKU if it exhibits an increase of at least 0.05 μg/mL of cinnamic acid produced by the enzyme variant as compared to the wild-type PAL enzyme following thermal challenge. A variant may be selected for use to degrade phenylalanine or to treat PKU if it shows an increase of at least 10% (e.g., 15%, 20%, 25%, 30%, 35%, 40%, 45%, 50%, 55%, 60%, 65%, 70% 75%, 80%, 85%, 90%, 95%, 100% or more) residual activity following thermal challenge relative to wild-type PAL enzyme. The residual PAL activity may be calculated by first measuring the PAL activity of the enzyme prior to thermal challenge and second measuring the PAL activity of the enzyme following thermal challenge and dividing the latter by the former. The amount of PA activity may be detected by measuring the amount of cinnamic acid measure via liquid chromatography with UV detection following a 30-minute incubation period of 25 μL of 0.7 to 1 μg/μL unchallenged and thermal challenged enzyme with 25 μL of 80 mM phenylalanine.

At least one mutation that increases the residual PAL activity following physicochemical stress may be introduced into the variant, allow the variant to remain active and degrade phenylalanine following application of the physicochemical stress as compared to the wild-type PAL enzyme. One such physicochemical stress may be nebulization, wherein the enzyme in a liquid sample is converted into a mist. The nebulization challenge assay may include loading 3 mL of a liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample. A variant may be selected for use to degrade phenylalanine or to treat PKU if it shows an increase of at least 10% (e.g., 15%, 20%, 25%, 30%, 35%, 40%, 45%, 50%, 55%, 60%, 65%, 70% 75%, 80%, 85%, 90%, 95%, 100% or more) residual activity following thermal challenge relative to wild-type PAL enzyme. The residual PAL activity may be calculated by first measuring the PAL activity of the enzyme prior to nebulization and second measuring the PAL activity of the enzyme following nebulization challenge and dividing the latter by the former. PAL activity is calculated by determining the amount of cinnamic acid produced via liquid chromatography with UV detection following a 30-minute incubation of 25 μL of 0.7 to 1 g/L of unchallenged and nebulization challenged enzyme with 25 μL of 80 mM phenylalanine.

At least one mutation that increases the catalytic activity of the enzyme may be introduced into the variant, allowing the variant to more rapidly and/or more efficiently break-down phenylalanine. Such a mutation may improve various measures of enzymatic performance, including but not limited to, increasing k_{cat}, lowering K_{M}, increasing k_{cat}/K_{M}, and/or increasing Vₘₐₓ. Such a mutation may also increase the amount of phenylalanine degraded by the PAL variant and increase the production of various phenylalanine degradation products, such as, but not limited to, cinnamic acid. Such a mutation may allow the protein to refold completely and faster after the stress challenge.

As noted above, the PAL enzyme variants may exhibit increased PAL activity, increased thermostability, increased residual enzyme activity following nebulization, and/or decreased immunogenicity relative to the wild-type PAL enzyme. The variants may comprise one or more mutations to the amino acid sequence of wild-type PAL encoding gene as disclosed in SEQ ID NO: 1, including one or more deletions, additions, or substitutions. (SEQ ID NO: 1—MKTLSQAQSKTSSQQFSFTGNSSANVIIGNQKLTINDVARVARNGTLVSLTNNTDILQGI QASCDYINNAVESGEPIYGVTSGFGGMANVAISREQASELQTNLVWFLKTGAGNKLPLA DVRAAMLLRANSHMRGASGIRLELIKRMEIFLNAGVTPYVYEFGSIGASGDLVPL SYITG SLIGLDPSFKVDFNGKEMDAPTALRQLNLSPLTLLPKEGLAMMNGTSVMTGIAANCVY DTQILTAIAMGVHALDIQALNGTNQSFHPFIHNSKPHPGQLWAADQMISLLANSQLVRD ELDGKHDYRDHELIQDRYSLRCLPQYLGPIVDGISQIAKQIEIEINSVTDNPLIDVDNQAS YHGGNFLGQYVGMGMDHLRYYIGLLAKHLDVQIALLASPEFSNGLPPSLLGNRERKVN MGLKGLQICGNSIMPLLTFYGNSIADRFPTHAEQFNQNINSQGYTSATLARRSVDIFQNY VAIALMFGVQAVDLRTYKKTGHYDARACLSPATERLYSAVRHVVGQKPTSDRPYIWN DNEQGLDEHIARISADIAAGGVIVQAVQDILPCLH).

The PAL enzyme variants may include one or more mutations in regions outside of the active site of the wild-type PAL enzyme relevant to its phenylalanine degrading activity, such as a mutation at one or more positions selected from any one of amino acid residues 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1. For the purposes of the present disclosure, the N-terminal methionine residue (M) of SEQ ID NO:1 may be cleaved off in the purified protein, but all amino acid position designations disclosed herein with reference to enzyme variants include the methionine residue for the purpose of maintaining amino acid numbering conventions. The variant may include one, two, three, four, five, six, seven, eight, nine, ten, or more substitutions. The variant may include one, two, three, four, five, six, seven, eight, nine, ten, eleven, twelve, thirteen, fourteen, or more substitutions at positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1.

The disclosed PAL enzyme variants can comprise at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1. The PAL enzyme variant sequence may include at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D. The PAL enzyme variant sequence may be any one of SEQ ID NOs: 2-41.

The PAL enzyme variant sequence may include at least two substitutions at two or more amino acid positions selected from 25, 30, 56, 64, 70, 96, 104, 247, 285, 457, 461, 483, 503, and 521 of SEQ ID NO: 1. The PAL enzyme variant sequence may include at least three substitutions at three or more amino acid positions selected from 25, 30, 56, 64, 70, 96, 104, 247, 285, 457, 461, 483, 503, and 521 of SEQ ID NO: 1. The PAL enzyme variant sequence may include at least three substitutions selected from N25E, N30E, I56V, C64V, A70S, Q96E, L104M, M247L, I285L, Q457M, S461A, G483A, C503T, and Q521V. The PAL enzyme variant sequence may include at least three substitutions, wherein the combination of at least three substitutions is selected from: a) A70S, Q96E, G483A; b) Q96E, G483A, C503T; c) A70S, G483A, C503T; d) I56V, M247L, Q457M; e) I56V, Q457M, Q521V; f) I56V, S461A, Q521V; g) N30E, Q96E, I285L; h) N30E, Q96E, C503T; and i) C64V, L104M, C503T. The PAL enzyme variant sequence may be any one of SEQ ID NOs: 42-47.

The PAL enzyme variant sequence may include at least four substitutions at four or more amino acid positions selected from 25, 30, 64, 70, 96, 104, 247, 285, 457, 461, 483, 503, and 521 of SEQ ID NO: 1. The PAL enzyme variant may include at least four substitutions selected from N25E, N30E, C64V, A70S, Q96E, L104M, M247L I285L, Q457M, S461A, G483A, C503T, and Q521V. The PAL enzyme variant sequence may include at least four substitutions, wherein the combination of at least four substitutions is selected from: a) A70S, Q96E, G483A, C503T; b) N25E, C64V, M247L, Q457M; c) N25E, C64V, M247L, S461A; d) N30E, Q96E, I285L, C503T; e) C64V, L104M, I285L, Q521V; and f) N30E, L104, 1285L, Q521V. The PAL enzyme variant sequence may be any one of SEQ ID NOs: 48-52.

The PAL enzyme variant sequence may include at least five substitutions at five or more amino acid positions selected from 30, 64, 70, 96, 104, 483, 503, and 521 of SEQ ID NO: 1. The PAL enzyme variant may include at least five substitutions selected from N30E, C64V, A70S, Q96E, L104M, G483A, C503T, and Q521V. The PAL enzyme variant sequence may include at least five substitutions, wherein the combination of at least five substitutions is selected from: a) A70S, Q96E, G483A, C503T, N30E; b) A70S, Q96E, G483A, C503T, L104M; c) L104M, C64V, N30E, G483A, C503T; d) Q96E, C64V, N30E, G483A, C503T; e) L104M, C64V, N30E, G483A, Q521V; and f) Q96E, C64V, N30E, G483A, Q521V. The PAL enzyme variant sequence may be any one of SEQ ID NOs: 53-58.

The enzyme variant sequence may include at least six substitutions at six or more amino acid positions selected from 30, 64, 70, 96, 104, 483, 503, and 521 of SEQ ID NO: 1. The PAL enzyme variant may include at least six substitutions selected from N30E, C64V, A70S, Q96E, L104M, G483A, C503T, and Q521V. The PAL enzyme variant sequence may include at least six substitutions, wherein the combination of at least six substitutions is selected from: a) A70S, Q96E, G483A, C503T, I285L, Q521V; b) Q96E, C64V, N30E, G483A, C503T, Q521V; and c) L104M, C64V, N30E, G483A, C503T, Q521V. The PAL enzyme variant sequence may be any one of SEQ ID NOs: 59-61.

The enzyme variant sequence may include at least seven substitutions at seven or more amino acid positions selected from 30, 64, 70, 96, 104, 483, 503, and 521 of SEQ ID NO: 1. The enzyme variant sequence may include at least eight substitutions at eight or more amino acid positions selected from 30, 64, 70, 96, 104, 483, 503, and 521 of SEQ ID NO: 1. The PAL enzyme variant may include at least eight substitutions selected from N30E, C64V, A70S, Q96E, L104M, G483A, C503T, and Q521V. The PAL enzyme variant sequence may include at least eight substitutions, wherein the combination of at least eight substitutions is N30E, C64V, A70S, Q96E, L104M, G483A, C503T, Q521V. The PAL enzyme variant sequence may be any one of SEQ ID NOs: 62. The enzyme variant sequence may include at least nine, ten, eleven, twelve, thirteen, fourteen, or more substitutions at least nine, ten, eleven, twelve, thirteen, fourteen, or more amino acid positions.

The amino acid sequence of exemplary variants of PAL are set forth in Table 1 below. These sequences are merely examples of variants within the scope of the disclosure.

TABLE 1
Examples of PAL Variants

| Enzyme | SEQ ID NO. | Sequence |
|---|---|---|
| SDPAL-01 | 2 | MKTLSQAQSKTSSQQFSFTGNSSAEVIIGNQKLTINDVA |
| (N25E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-02 | 3 | MKTLSQAQSKTSSQQFSFTGNSSANVIIGEQKLTINDVA |
| (N30E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-03 | 4 | MKTLSQAQSKTSSQQFSFTGNSSANVIIGNQKLTINDVA |
| (N44E) | | RVAREGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-04 | | MKTLSQAQSKTSSQQFSFTGNSSANVIIGNQKLTINDVA |
| (T46V) | | RVARNGVLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-05 | 6 | MKTLSQAQSKTSSQQFSFTGNSSANVIIGNQKLTINDVA |
| (I56V) | | RVARNGTLVSLTNNTDVLQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-07 | 7 | MKTLSQAQSKTSSQQFSFTGNSSANVIIGNQKLTINDVA |
| (C64V) | | RVARNGTLVSLTNNTDILQGIQASVDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-09 | 8 | MKTLSQAQSKTSSQQFSFTGNSSANVIIGNQKLTINDVA |
| (Q96E) | | RVARNGTLVSLTNNTDILQGIQASCDYINNAVESGEPIY |
| | | GVTSGFGGMANVAISREEASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-10 | 9 | MKTLSQAQSKTSSQQFSFTGNSSANVIIGNQKLTINDVA |
| (L104M) | | RVARNGTLVSLTNNTDILQGIQASCDYINNAVESGEPIY |
| | | GVTSGFGGMANVAISREQASELQTNMVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-11 | 10 | MKTLSQAQSKTSSQQFSFTGNSSANVIIGNQKLTINDVA |
| (T110V) | | RVARNGTLVSLTNNTDILQGIQASCDYINNAVESGEPIY |
| | | GVTSGFGGMANVAISREQASELQTNLVWFLKVGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-12 | 11 | MKTLSQAQSKTSSQQFSFTGNSSANVIIGNQKLTINDVA |
| (K115Y) | | RVARNGTLVSLTNNTDILQGIQASCDYINNAVESGEPIY |
| | | GVTSGFGGMANVAISREQASELQTNLVWFLKTGAGNY |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-15 | 12 | MKTLSQAQSKTSSQQFSFTGNSSANVIIGNQKLTINDVA |
| (N234L) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAALCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-16 | 13 | MKTLSQAQSKTSSQQFSFTGNSSANVIIGNQKLTINDVA |
| (M247L) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIALGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-17 | 14 | MKTLSQAQSKTSSQQFSFTGNSSANVIIGNQKLTINDVA |
| (D253H) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALHIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSA VRHVVGQKPTSDRPYIWNDNEQG |
| | | LDEHIARISADIAAGGVIVQAVQDILPCLH |
| SDPAL-19 | 15 | MKTLSQAQSKTSSQQFSFTGNSSANVIIGNQKLTINDVA |
| (I285L) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGQKPTSDRPYIWNDNEQ |
| | | GLDEHIARISADIAAGGVIVQAVQDILPCLH |
| SDPAL-20 | 16 | MKTLSQAQSKTSSQQFSFTGNSSANVIIGNQKLTINDVA |
| (L316I) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSIRCLPQYLGPIVDGISQIAKQIEIEINSVTDNP |
| | | LIDVDNQASYHGGNFLGQYVGMGMDHLRYYIGLLAKH |
| | | LDVQIALLASPEFSNGLPPSLLGNRERKVNMGLKGLQIC |
| | | GNSIMPLLTFYGNSIADRFPTHAEQFNQNINSQGYTSAT |
| | | LARRSVDIFQNYVAIALMFGVQAVDLRTYKKTGHYDA |
| | | RACLSPATERLYSAVRHVVGQKPTSDRPYIWNDNEQGL |
| | | DEHIARISADIAAGGVIVQAVQDILPCLH |
| SDPAL-23 | 17 | MKTLSQAQSKTSSQQFSFTGNSSANVIIGNQKLTINDVA |
| (Q389N) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVNIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-24 | 18 | MKTLSQAQSKTSSQQFSFTGNSSANVIIGNQKLTINDVA |
| (M416Q) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNQGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-26 | 19 | MKTLSQAQSKTSSQQFSFTGNSSANVIIGNQKLTINDVA |
| (C424T) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | TGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-27 | 20 | MKTLSQAQSKTSSQQFSFTGNSSANVIIGNQKLTINDVA |
| (N437E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGESIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-28 | 21 | MKTLSQAQSKTSSQQFSFTGNSSANVIIGNQKLTINDVA |
| (A440S) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSISDRFPTHAEQFNQNINSQGYTSAT |
| | | LARRSVDIFQNYVAIALMFGVQAVDLRTYKKTGHYDA |
| | | RACLSPATERLYSAVRHVVGQKPTSDRPYIWNDNEQGL |
| | | DEHIARISADIAAGGVIVQAVQDILPCLH |
| SDPAL-29 | 22 | MKTLSQAQSKTSSQQFSFTGNSSANVIIGNQKLTINDVA |
| (E448D) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHADQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-30 | 23 | MKTLSQAQSKTSSQQFSFTGNSSANVIIGNQKLTINDVA |
| (Q449E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEEFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-31 | 24 | MKTLSQAQSKTSSQQFSFTGNSSANVIIGNQKLTINDVA |
| (Q457M) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSMGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-32 | 25 | MKTLSQAQSKTSSQQFSFTGNSSANVIIGNQKLTINDVA |
| (S461A) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTAA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-34 | 26 | MKTLSQAQSKTSSQQFSFTGNSSANVIIGNQKLTINDVA |
| (N474V) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQVYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-35 | 27 | MKTLSQAQSKTSSQQFSFTGNSSANVIIGNQKLTINDVA |
| (G483A) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL 37 | 28 | MKTLSQAQSKTSSQQFSFTGNSSANVIIGNQKLTINDVA |
| (C503T) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARATLSPATERLYSAVRHVVGQKPTSDRPYIWNDNEQG |
| | | LDEHIARISADIAAGGVIVQAVQDILPCLH |
| SDPAL-38 | 29 | MKTLSQAQSKTSSQQFSFTGNSSANVIIGNQKLTINDVA |
| (Q521V) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGVKPTSDRPYIWNDNEQG |
| | | LDEHIARISADIAAGGVIVQAVQDILPCLH |
| SDPAL-41 | 30 | MKTLSQAQSKTSSQQFSFTGNSSANVIIGNQKLTINDVA |
| (N534D) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDDEQG |
| | | LDEHIARISADIAAGGVIVQAVQDILPCLH |
| SDPAL-42 | 31 | MKTLSQAQSKTSSQQFSFTGNSSANVVIGNQKLTINDV |
| (I27V) | | ARVARNGTLVSLTNNTDILQGIQASCDYINNAVESGEPI |
| | | YGVTSGFGGMANVAISREQASELQTNLVWFLKTGAGN |
| | | KLPLADVRAAMLLRANSHMRGASGIRLELIKRMEIFLN |
| | | AGVTPYVYEFGSIGASGDLVPLSYITGSLIGLDPSFKVDF |
| | | NGKEMDAPTALRQLNLSPLTLLPKEGLAMMNGTSVMT |
| | | GIAANCVYDTQILTAIAMGVHALDIQALNGTNQSFHPFI |
| | | HNSKPHPGQLWAADQMISLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGQKPTSDRPYIWNDNEQ |
| | | GLDEHIARISADIAAGGVIVQAVQDILPCLHG |
| SDPAL-43 | 32 | MKTLSQAQSKTSSQQFSFTGNSSANVIIGNRKLTINDVA |
| (Q31R) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-45 | 33 | MKTLSQAQSKTSSQQFSFTGNSSANVIIGNQKLTINDVA |
| (L47S) | | RVARNGTSVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-48 | 34 | MKTLSQAQSKTSSQQFSFTGNSSANVIIGNQKLTINDVA |
| (K115L) | | RVARNGTLVSLTNNTDILQGIQASCDYINNAVESGEPIY |
| | | GVTSGFGGMANVAISREQASELQTNLVWFLKTGAGNL |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-49 | 35 | MKTLSQAQSKTSSQQFSFTGNSSANVIIGNQKLTINDVA |
| (L118K) | | RVARNGTLVSLTNNTDILQGIQASCDYINNAVESGEPIY |
| | | GVTSGFGGMANVAISREQASELQTNLVWFLKTGAGNK |
| | | LPKADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-50 | 36 | MKTLSQAQSKTSSQQFSFTGNSSANVIIGNQKLTINDVA |
| (R134K) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMKGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-51 | 37 | MKTLSQAQSKTSSQQFSFTGNSSANVIIGNQKLTINDVA |
| (S209A) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLAPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-54 | 38 | MKTLSQAQSKTSSQQFSFTGNSSANVIIGNQKLTINDVA |
| (A246S) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAISMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-55 | 39 | MKTLSQAQSKTSSQQFSFTGNSSANVIIGNQKLTINDVA |
| (D282A) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAAAQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-56 | 40 | MKTLSQAQSKTSSQQFSFTGNSSANVIIGNQKLTINDVA |
| (I310V) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELVQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGQKPTSDRPYIWNDNEQ |
| | | GLDEHIARISADIAAGGVIVQAVQDILPCLHG |
| SDPAL-58 | 41 | MKTLSQAQSKTSSQQFSFTGNSSANVIIGNQKLTINDVA |
| (R313K) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDKYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| RDPAL-02 | 42 | MKTLSQAQSKTSSQQFSFTGNSSANVIIGNQKLTINDVA |
| (Q96E, | | RVARNGTLVSLTNNTDILQGIQASCDYINNAVESGEPIY |
| G483A, | | GVTSGFGGMANVAISREEASELQTNLVWFLKTGAGNK |
| C503T) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-16 | 43 | MKTLSQAQSKTSSQQFSFTGNSSANVIIGNQKLTINDVA |
| (I56V, | | RVARNGTLVSLTNNTDVLQGIQASCDYINNAVESGEPIY |
| Q457M, | | GVTSGFGGMANVAISREQASELQTNLVWFLKTGAGNK |
| Q521V) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSMGYTSA |
| | | TLARRSVDIFQNYVAIALMFGVQAVDLRTYKKTGHYD |
| | | ARACLSPATERLYSAVRHVVGVKPTSDRPYIWNDNEQG |
| | | LDEHIARISADIAAGGVIVQAVQDILPCLHG |
| RDPAL-17 | 44 | MKTLSQAQSKTSSQQFSFTGNSSANVIIGNQKLTINDVA |
| (I56V, | | RVARNGTLVSLTNNTDVLQGIQASCDYINNAVESGEPIY |
| S461A, | | GVTSGFGGMANVAISREQASELQTNLVWFLKTGAGNK |
| Q521V) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTAA |
| | | TLARRSVDIFQNYVAIALMFGVQAVDLRTYKKTGHYD |
| | | ARACLSPATERLYSAVRHVVGVKPTSDRPYIWNDNEQG |
| | | LDEHIARISADIAAGGVIVQAVQDILPCLHG |
| RDPAL-20 | 45 | MKTLSQAQSKTSSQQFSFTGNSSANVIIGEQKLTINDVA |
| (N30E, | | RVARNGTLVSLTNNTDILQGIQASCDYINNAVESGEPIY |
| Q96E, | | GVTSGFGGMANVAISREEASELQTNLVWFLKTGAGNK |
| I285L) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGQKPTSDRPYIWNDNEQ |
| | | GLDEHIARISADIAAGGVIVQAVQDILPCLHG |
| RDPAL-21 | 46 | MKTLSQAQSKTSSQQFSFTGNSSANVIIGEQKLTINDVA |
| (N30E, | | RVARNGTLVSLTNNTDILQGIQASCDYINNAVESGEPIY |
| Q96E, | | GVTSGFGGMANVAISREEASELQTNLVWFLKTGAGNK |
| C503T) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-22 | 47 | MKTLSQAQSKTSSQQFSFTGNSSANVIIGNQKLTINDVA |
| (C64V, | | RVARNGTLVSLTNNTDILQGIQASVDYINNAVESGEPIY |
| L104M, | | GVTSGFGGMANVAISREQASELQTNMVWFLKTGAGNK |
| C503T) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-18 | 48 | MKTLSQAQSKTSSQQFSFTGNSSAEVIIGNQKLTINDVA |
| (N25E, | | RVARNGTLVSLTNNTDILQGIQASVDYINNAVESGEPIY |
| C64V, | | GVTSGFGGMANVAISREQASELQTNLVWFLKTGAGNK |
| M247L, | | LPLADVRAAMLLRANSHMRGASGIRLELIKRMEIFLNA |
| Q457M) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIALGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSMGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| RDPAL-19 | 49 | MKTLSQAQSKTSSQQFSFTGNSSAEVIIGNQKLTINDVA |
| (N25E, | | RVARNGTLVSLTNNTDILQGIQASVDYINNAVESGEPIY |
| C64V, | | GVTSGFGGMANVAISREQASELQTNLVWFLKTGAGNK |
| M247L, | | LPLADVRAAMLLRANSHMRGASGIRLELIKRMEIFLNA |
| S461A) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIALGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTAA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| RDPAL-23 | 50 | MKTLSQAQSKTSSQQFSFTGNSSANVIIGEQKLTINDVA |
| (N30E, | | RVARNGTLVSLTNNTDILQGIQASCDYINNAVESGEPIY |
| Q96E, | | GVTSGFGGMANVAISREEASELQTNLVWFLKTGAGNK |
| I285L, | | LPLADVRAAMLLRANSHMRGASGIRLELIKRMEIFLNA |
| C503T) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARATLSPATERLYSAVRHVVGQKPTSDRPYIWNDNEQ |
| | | GLDEHIARISADIAAGGVIVQAVQDILPCLHG |
| RDPAL-24 | 51 | MKTLSQAQSKTSSQQFSFTGNSSANVIIGNQKLTINDVA |
| (C64V, | | RVARNGTLVSLTNNTDILQGIQASVDYINNAVESGEPIY |
| L104M, | | GVTSGFGGMANVAISREQASELQTNMVWFLKTGAGNK |
| I285L, | | LPLADVRAAMLLRANSHMRGASGIRLELIKRMEIFLNA |
| Q521V) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGVKPTSDRPYIWNDNEQ |
| | | GLDEHIARISADIAAGGVIVQAVQDILPCLHG |
| RPDAL-25 | 52 | MKTLSQAQSKTSSQQFSFTGNSSANVIIGEQKLTINDVA |
| (N30E, | | RVARNGTLVSLTNNTDILQGIQASCDYINNAVESGEPIY |
| L104M, | | GVTSGFGGMANVAISREQASELQTNMVWFLKTGAGNK |
| I285L, | | LPLADVRAAMLLRANSHMRGASGIRLELIKRMEIFLNA |
| Q521V) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFGVQAVDLRTYKKTGHY |
| | | DARACLSPATERLYSAVRHVVGVKPTSDRPYIWNDNEQ |
| | | GLDEHIARISADIAAGGVIVQAVQDILPCLHG |
| RDPAL-05 | 53 | MKTLSQAQSKTSSQQFSFTGNSSANVIIGEQKLTINDVA |
| (A70S, | | RVARNGTLVSLTNNTDILQGIQASCDYINNSVESGEPIY |
| Q96E, | | GVTSGFGGMANVAISREEASELQTNLVWFLKTGAGNK |
| G483A, | | LPLADVRAAMLLRANSHMRGASGIRLELIKRMEIFLNA |
| C503T, | | GVTPYVYEFGSIGASGDLVPLSYITGSLIGLDPSFKVDFN |
| N30E) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-06 | 54 | MKTLSQAQSKTSSQQFSFTGNSSANVIIGNQKLTINDVA |
| (A70S, | | RVARNGTLVSLTNNTDILQGIQASCDYINNSVESGEPIY |
| Q96E, | | GVTSGFGGMANVAISREEASELQTNMVWFLKTGAGNK |
| G483A, | | LPLADVRAAMLLRANSHMRGASGIRLELIKRMEIFLNA |
| C503T, | | GVTPYVYEFGSIGASGDLVPLSYITGSLIGLDPSFKVDFN |
| L104M) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-11 | 55 | MKTLSQAQSKTSSQQFSFTGNSSANVIIGEQKLTINDVA |
| (L104M, | | RVARNGTLVSLTNNTDILQGIQASVDYINNAVESGEPIY |
| C64V, | | GVTSGFGGMANVAISREQASELQTNMVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| C503T) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-12 | 56 | MKTLSQAQSKTSSQQFSFTGNSSANVIIGEQKLTINDVA |
| (Q96E, | | RVARNGTLVSLTNNTDILQGIQASVDYINNAVESGEPIY |
| C64V, | | GVTSGFGGMANVAISREEASELQTNLVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| C503T) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLHG |
| RDPAL-13 | 57 | MKTLSQAQSKTSSQQFSFTGNSSANVIIGEQKLTINDVA |
| (L104M, | | RVARNGTLVSLTNNTDILQGIQASVDYINNAVESGEPIY |
| C64V, | | GVTSGFGGMANVAISREQASELQTNMVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| Q521V) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARACLSPATERLYSAVRHVVGVKPTSDRPYIWNDNEQG |
| | | LDEHIARISADIAAGGVIVQAVQDILPCLHG |
| RDPAL-14 | 58 | MKTLSQAQSKTSSQQFSFTGNSSANVIIGEQKLTINDVA |
| (Q96E, | | RVARNGTLVSLTNNTDILQGIQASVDYINNAVESGEPIY |
| C64V, | | GVTSGFGGMANVAISREEASELQTNLVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| Q521V) | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARACLSPATERLYSAVRHVVGVKPTSDRPYIWNDNEQG |
| | | LDEHIARISADIAAGGVIVQAVQDILPCLHG |
| RDPAL-07 | 59 | MKTLSQAQSKTSSQQFSFTGNSSANVIIGNQKLTINDVA |
| (A70S, | | RVARNGTLVSLTNNTDILQGIQASCDYINNSVESGEPIY |
| Q96E, | | GVTSGFGGMANVAISREEASELQTNLVWFLKTGAGNK |
| G483A, | | LPLADVRAAMLLRANSHMRGASGIRLELIKRMEIFLNA |
| C503T, | | GVTPYVYEFGSIGASGDLVPLSYITGSLIGLDPSFKVDFN |
| I285L, | | GKEMDAPTALRQLNLSPLTLLPKEGLAMMNGTSVMTG |
| Q521V) | | IAANCVYDTQILTAIAMGVHALDIQALNGTNQSFHPFIH |
| | | NSKPHPGQLWAADQMLSLLANSQLVRDELDGKHDYR |
| | | DHELIQDRYSLRCLPQYLGPIVDGISQIAKQIEIEINSVTD |
| | | NPLIDVDNQASYHGGNFLGQYVGMGMDHLRYYIGLLA |
| | | KHLDVQIALLASPEFSNGLPPSLLGNRERKVNMGLKGL |
| | | QICGNSIMPLLTFYGNSIADRFPTHAEQFNQNINSQGYTS |
| | | ATLARRSVDIFQNYVAIALMFAVQAVDLRTYKKTGHY |
| | | DARATLSPATERLYSAVRHVVGVKPTSDRPYIWNDNEQ |
| | | GLDEHIARISADIAAGGVIVQAVQDILPCLHG |
| RDPAL-09 | 60 | MKTLSQAQSKTSSQQFSFTGNSSANVIIGEQKLTINDVA |
| (Q96E, | | RVARNGTLVSLTNNTDILQGIQASVDYINNAVESGEPIY |
| C64V, | | GVTSGFGGMANVAISREEASELQTNLVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| C503T, | | GKEMDAPTALRQLNLSPLTLLPKEGLAMMNGTSVMTG |
| Q521V) | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGVKPTSDRPYIWNDNEQG |
| | | LDEHIARISADIAAGGVIVQAVQDILPCLHG |
| RDPAL-10 | 61 | MKTLSQAQSKTSSQQFSFTGNSSANVIIGEQKLTINDVA |
| (L104M, | | RVARNGTLVSLTNNTDILQGIQASVDYINNAVESGEPIY |
| C64V, | | GVTSGFGGMANVAISREQASELQTNMVWFLKTGAGNK |
| N30E, | | LPLADVRAAMLLRANSHMRGASGIRLELIKRMEIFLNA |
| G483A, | | GVTPYVYEFGSIGASGDLVPLSYITGSLIGLDPSFKVDFN |
| C503T, | | GKEMDAPTALRQLNLSPLTLLPKEGLAMMNGTSVMTG |
| Q521V) | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGVKPTSDRPYIWNDNEQG |
| | | LDEHIARISADIAAGGVIVQAVQDILPCLHG |
| RDPAL-08 | 62 | MKTLSQAQSKTSSQQFSFTGNSSANVIIGEQKLTINDVA |
| (N30E, | | RVARNGTLVSLTNNTDILQGIQASVDYINNSVESGEPIY |
| C64V, | | GVTSGFGGMANVAISREEASELQTNMVWFLKTGAGNK |
| A70S, | | LPLADVRAAMLLRANSHMRGASGIRLELIKRMEIFLNA |
| Q96E, | | GVTPYVYEFGSIGASGDLVPLSYITGSLIGLDPSFKVDFN |
| L104M, | | GKEMDAPTALRQLNLSPLTLLPKEGLAMMNGTSVMTG |
| G483A, | | IAANCVYDTQILTAIAMGVHALDIQALNGTNQSFHPFIH |
| C503T, | | NSKPHPGQLWAADQMISLLANSQLVRDELDGKHDYRD |
| Q521V) | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGVKPTSDRPYIWNDNEQG |
| | | LDEHIARISADIAAGGVIVQAVQDILPCLHG |
| SDPAL-06 | 63 | MKTLSQAQSKTSSQQFSFTGNSSANVIIGNQKLTINDVA |
| (I56T) | | RVARNGTLVSLTNNTDTLQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SPDAL-08 | 64 | MKTLSQAQSKTSSQQFSFTGNSSANVIIGNQKLTINDVA |
| (A70S) | | RVARNGTLVSLTNNTDILQGIQASCDYINNSVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNREKVNMGLKGLQIC |
| | | GNSIMPLLTFYGNSIADRFPTHAEQFNQNINSQGYTSAT |
| | | LARRSVDIFQNYVAIALMFGVQAVDLRTYKKTGHYDA |
| | | RACLSPATERLYSAVRHVVGQKPTSDRPYIWNDNEQGL |
| | | DEHIARISADIAAGGVIVQAVQDILPCLH |
| SDPAL-13 | 65 | MKTLSQAQSKTSSQQFSFTGNSSANVIIGNQKLTINDVA |
| (Y160K) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVKEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-14 | 66 | MKTLSQAQSKTSSQQFSFTGNSSANVIIGNQKLTINDVA |
| (F188Y) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSYKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-18 | 67 | MKTLSQAQSKTSSQQFSFTGNSSANVIIGNQKLTINDVA |
| (D253F) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALFIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-21 | 68 | MKTLSQAQSKTSSQQFSFTGNSSANVIIGNQKLTINDVA |
| (N347D) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDD |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-22 | 69 | MKTLSQAQSKTSSQQFSFTGNSSANVIIGNQKLTINDVA |
| (Q366M) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGMYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-25 | 70 | MKTLSQAQSKTSSQQFSFTGNSSANVIIGNQKLTINDVA |
| (Q422E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVNIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-33 | 71 | MKTLSQAQSKTSSQQFSFTGNSSANVIIGNQKLTINDVA |
| (Q473K) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFKNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| SDPAL-36 | 72 | MKTLSQAQSKTSSQQFSFTGNSSANVIIGNQKLTINDVA |
| (G483S) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFSVQAVDLRTYKKTGHYDA |
| | | RACLSPATERLYSAVRHVVGQKPTSDRPYIWNDNEQGL |
| | | DEHIARISADIAAGGVIVQAVQDILPCLH |
| SDPAL-39 | 73 | MKTLSQAQSKTSSQQFSFTGNSSANVIIGNQKLTINDVA |
| | | RVARNGTLVSLTNNTDILQGIQASCDYINNAVESGEPIY |
| (Q521E) | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGEKPTSDRPYIWNDNEQG |
| | | LDEHIARISADIAAGGVIVQAVQDILPCLH |
| SDPAL-40 | 74 | MKTLSQAQSKTSSQQFSFTGNSSANVIIGNQKLTINDVA |
| (Y529F) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPFIWNDNEQG |
| | | LDEHIARISADIAAGGVIVQAVQDILPCLH |
| SDPAL-44 | 75 | MKTLSQAQSKTSSQQFSFTGNSSANVIIGNQKLTINDVA |
| (R43Y) | | RVAYNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-46 | 76 | MKTLSQAQSKTSSQQFSFTGNSSANVIIGNQKLTINDVA |
| (E72K) | | RVARNGTLVSLTNNTDILQGIQASCDYINNAVKSGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-47 | 77 | MKTLSQAQSKTSSQQFSFTGNSSANVIIGNQKLTINDVA |
| (192D) | | RVARNGTLVSLTNNTDILQGIQASCDYINNAVESGEPIY |
| | | GVTSGFGGMANVADSREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-52 | 78 | MKTLSQAQSKTSSQQFSFTGNSSANVIIGNQKLTINDVA |
| (K216R) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPREGLAMMNGTSVMTGI |
| | | AANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-53 | 79 | MKTLSQAQSKTSSQQFSFTGNSSANVIIGNQKLTINDVA |
| (N223E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMEGTSVMTGI |
| | | AANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-57 | 80 | MKTLSQAQSKTSSQQFSFTGNSSANVIIGNQKLTINDVA |
| (Q311E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIEDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| SDPAL-59 | 81 | MKTLSQAQSKTSSQQFSFTGNSSANVIIGNQKLTINDVA |
| (Q321E) | | RVARNGTLVSLTNNTDILQGIQASCDYINNAVESGEPIY |
| | | GVTSGFGGMANVAISREQASELQTNLVWFLKTGAGNK |
| | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPEYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLHG |
| RDPAL-01 | 82 | MKTLSQAQSKTSSQQFSFTGNSSANVIIGNQKLTINDVA |
| (A70S, | | RVARNGTLVSLTNNTDILQGIQASCDYINNSVESGEPIY |
| Q96E, | | GVTSGFGGMANVAISREEASELQTNLVWFLKTGAGNK |
| G483A) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARACLSPATERLYSAVRHVVGQKPTSDRPYIWNDNEQG |
| | | LDEHIARISADIAAGGVIVQAVQDILPCLH |
| RDPAL-03 | 83 | MKTLSQAQSKTSSQQFSFTGNSSANVIIGNQKLTINDVA |
| (A70S, | | RVARNGTLVSLTNNTDILQGIQASCDYINNSVESGEPIY |
| G483A, | | GVTSGFGGMANVAISREQASELQTNLVWFLKTGAGNK |
| C503T) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLH |
| RDPAL-04 | 84 | MKTLSQAQSKTSSQQFSFTGNSSANVIIGNQKLTINDVA |
| (A70S, | | RVARNGTLVSLINNTDILQGIQASCDYINNSVESGEPIY |
| Q96E, | | GVTSGFGGMANVAISREEASELQTNLVWFLKTGAGNK |
| G483A, | | LPLADVRAAMLLRANSHMRGASGIRLELIKRMEIFLNA |
| C503T) | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIAMGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSQGYTSA |
| | | TLARRSVDIFQNYVAIALMFAVQAVDLRTYKKTGHYD |
| | | ARATLSPATERLYSAVRHVVGQKPTSDRPYIWNDNEQG |
| | | LDEHIARISADIAAGGVIVQAVQDILPCLH |
| RDPAL-15 | 85 | MKTLSQAQSKTSSQQFSFTGNSSANVIIGNQKLTINDVA |
| (I56V, | | RVARNGTLVSLTNNTDVLQGIQASCDYINNAVESGEPIY |
| M247L, | | GVTSGFGGMANVAISREQASELQTNLVWFLKTGAGNK |
| Q457M) | | LPLADVRAAMLLRANSHMRGASGIRLELIKRMEIFLNA |
| | | GVTPYVYEFGSIGASGDLVPLSYITGSLIGLDPSFKVDFN |
| | | GKEMDAPTALRQLNLSPLTLLPKEGLAMMNGTSVMTG |
| | | IAANCVYDTQILTAIALGVHALDIQALNGTNQSFHPFIH |
| | | NSKPHPGQLWAADQMISLLANSQLVRDELDGKHDYRD |
| | | HELIQDRYSLRCLPQYLGPIVDGISQIAKQIEIEINSVTDN |
| | | PLIDVDNQASYHGGNFLGQYVGMGMDHLRYYIGLLAK |
| | | HLDVQIALLASPEFSNGLPPSLLGNRERKVNMGLKGLQI |
| | | CGNSIMPLLTFYGNSIADRFPTHAEQFNQNINSMGYTSA |
| | | TLARRSVDIFQNYVAIALMFGVQAVDLRTYKKTGHYD |
| | | ARACLSPATERLYSAVRHVVGQKPTSDRPYIWNDNEQG |
| | | LDEHIARISADIAAGGVIVQAVQDILPCLH |

The N-terminal methionine residue (M) of any of the foregoing sequences can be cleaved off in the purified product

In some implementations, a PAL enzyme variant as described herein may comprise, or consist of SEQ ID NO: 2. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97% about 98%, or about 99% sequence identity with the variant sequence of SEQ ID NO: 2.

In some implementations, a PAL enzyme variant as described herein may comprise, or consist of SEQ ID NO: 3. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 3.

In some implementations, a PAL enzyme variant as described herein may comprise, or consist of SEQ ID NO: 4. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 4.

In some implementations, a PAL enzyme variant as described herein may comprise, or consist of, SEQ ID NO: 5. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 5.

In some implementations, a PAL enzyme variant as described herein may comprise, or consist of SEQ ID NO: 6. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 6.

In some implementations, a PAL enzyme variant as described herein may comprise, or consist of SEQ ID NO: 7. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 7.

In some implementations, a PAL enzyme variant as described herein may comprise, or consist of SEQ ID NO: 8. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 8.

In some implementations, a PAL enzyme variant as described herein may comprise, or consist of SEQ ID NO: 9. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 9.

In some implementations, a PAL enzyme variant as described herein may comprise, or consist of SEQ ID NO: 10. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 10.

In some implementations, a PAL enzyme variant as described herein may comprise, or consist of SEQ ID NO: 11. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 11.

In some implementations, a PAL enzyme variant as described herein may comprise, or consist of SEQ ID NO: 12. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 12.

In some implementations, a PAL enzyme variant as described herein may comprise, or consist of SEQ ID NO: 13. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 13.

In some implementations, a PAL enzyme variant as described herein may comprise, or consist of SEQ ID NO: 14. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 14.

In some implementations, a PAL enzyme variant as described herein may comprise, or consist of SEQ ID NO: 15. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 15.

In some implementations, a PAL enzyme variant as described herein may comprise, or consist of SEQ ID NO: 16. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 16.

In some implementations, a PAL enzyme variant as described herein may comprise, or consist of SEQ ID NO: 17. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 17.

In some implementations, a PAL enzyme variant as described herein may comprise, or consist of SEQ ID NO: 18. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 18.

In some implementations, a PAL enzyme variant as described herein may comprise, or consist of SEQ ID NO: 19. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 19.

In some implementations, a PAL enzyme variant as described herein may comprise, or consist of SEQ ID NO: 20. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 20.

In some implementations, a PAL enzyme variant as described herein may comprise, or consist of SEQ ID NO: 21. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 21.

In some implementations, a PAL enzyme variant as described herein may comprise, or consist of SEQ ID NO: 22. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 22.

In some implementations, a PAL enzyme variant as described herein may comprise, or consist of SEQ ID NO: 23. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 23.

In some implementations, a PAL enzyme variant as described herein may comprise, or consist of SEQ ID NO: 24. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 24.

In some implementations, a PAL enzyme variant as described herein may comprise, or consist of SEQ ID NO: 25. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 25.

In some implementations, a PAL enzyme variant as described herein may comprise, or consist of SEQ ID NO: 26. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 26.

In some implementations, a PAL enzyme variant as described herein may comprise, or consist of SEQ ID NO: 27. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 27.

In some implementations, a PAL enzyme variant as described herein may comprise, or consist of SEQ ID NO: 28. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 28.

In some implementations, a PAL enzyme variant as described herein may comprise, or consist of SEQ ID NO: 29. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 29.

In some implementations, a PAL enzyme variant as described herein may comprise, or consist of SEQ ID NO: 30. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 30.

In some implementations, a PAL enzyme variant as described herein may comprise, or consist of SEQ ID NO: 31. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 31.

In some implementations, a PAL enzyme variant as described herein may comprise, or consist of SEQ ID NO: 32. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 32.

In some implementations, a PAL enzyme variant as described herein may comprise, or consist of SEQ ID NO: 33. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 33.

In some implementations, a PAL enzyme variant as described herein may comprise, or consist of SEQ ID NO: 34. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 34.

In some implementations, a PAL enzyme variant as described herein may comprise, or consist of SEQ ID NO: 35. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 35.

In some implementations, a PAL enzyme variant as described herein may comprise, or consist of SEQ ID NO: 36. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 36.

In some implementations, a PAL enzyme variant as described herein may comprise, or consist of SEQ ID NO: 37. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 37.

In some implementations, a PAL enzyme variant as described herein may comprise, or consist of SEQ ID NO: 38. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 38.

In some implementations, a PAL enzyme variant as described herein may comprise, or consist of SEQ ID NO: 39. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 39.

In some implementations, a PAL enzyme variant as described herein may comprise, or consist of SEQ ID NO: 40. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 40.

In some implementations, a PAL enzyme variant as described herein may comprise, or consist of SEQ ID NO: 41. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 41.

In some implementations, a PAL enzyme variant as described herein may comprise, or consist of SEQ ID NO: 42. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 42.

In some implementations, a PAL enzyme variant as described herein may comprise, or consist of SEQ ID NO: 43. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 43.

In some implementations, a PAL enzyme variant as described herein may comprise, or consist of SEQ ID NO: 44. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 44.

In some implementations, a PAL enzyme variant as described herein may comprise, or consist of SEQ ID NO: 45. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 45.

In some implementations, a PAL enzyme variant as described herein may comprise, or consist of SEQ ID NO: 46. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 46.

In some implementations, a PAL enzyme variant as described herein may comprise, or consist of SEQ ID NO: 47. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 47.

In some implementations, a PAL enzyme variant as described herein may comprise, or consist of SEQ ID NO: 48. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 48.

In some implementations, a PAL enzyme variant as described herein may comprise, or consist of SEQ ID NO: 49. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 49.

In some implementations, a PAL enzyme variant as described herein may comprise, or consist of SEQ ID NO: 50. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 50.

In some implementations, a PAL enzyme variant as described herein may comprise, or consist of SEQ ID NO: 51. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 51.

In some implementations, a PAL enzyme variant as described herein may comprise, or consist of SEQ ID NO: 52. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 52.

In some implementations, a PAL enzyme variant as described herein may comprise, or consist of SEQ ID NO: 53. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 53.

In some implementations, a PAL enzyme variant as described herein may comprise, or consist of SEQ ID NO: 54. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 54.

In some implementations, a PAL enzyme variant as described herein may comprise, or consist of SEQ ID NO: 55. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 55.

In some implementations, a PAL enzyme variant as described herein may comprise, or consist of SEQ ID NO: 56. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 56.

In some implementations, a PAL enzyme variant as described herein may comprise, or consist of SEQ ID NO: 57. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 57.

In some implementations, a PAL enzyme variant as described herein may comprise, or consist of SEQ ID NO: 58. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 58.

In some implementations, a PAL enzyme variant as described herein may comprise, or consist of SEQ ID NO: 59. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 59.

In some implementations, a PAL enzyme variant as described herein may comprise, or consist of SEQ ID NO: 60. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 60.

In some implementations, a PAL enzyme variant as described herein may comprise, or consist of SEQ ID NO: 61. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 61.

In some implementations, a PAL enzyme variant as described herein may comprise, or consist of SEQ ID NO: 62. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 62.

In some implementations, a PAL enzyme variant as described herein may comprise, or consist of SEQ ID NO: 63. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 63.

In some implementations, a PAL enzyme variant as described herein may comprise, or consist of SEQ ID NO: 64. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 64.

In some implementations, a PAL enzyme variant as described herein may comprise, or consist of SEQ ID NO: 65. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 65.

In some implementations, a PAL enzyme variant as described herein may comprise, or consist of SEQ ID NO: 66. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 66.

In some implementations, a PAL enzyme variant as described herein may comprise, or consist of SEQ ID NO: 67. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 67.

In some implementations, a PAL enzyme variant as described herein may comprise, or consist of SEQ ID NO: 68. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 68.

In some implementations, a PAL enzyme variant as described herein may comprise, or consist of SEQ ID NO: 69. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 69.

In some implementations, a PAL enzyme variant as described herein may comprise, or consist of SEQ ID NO: 70. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 70.

In some implementations, a PAL enzyme variant as described herein may comprise, or consist of SEQ ID NO: 71. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 71.

In some implementations, a PAL enzyme variant as described herein may comprise, or consist of SEQ ID NO: 72. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 72.

In some implementations, a PAL enzyme variant as described herein may comprise, or consist of SEQ ID NO: 73. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 73.

In some implementations, a PAL enzyme variant as described herein may comprise, or consist of SEQ ID NO: 74. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 74.

In some implementations, a PAL enzyme variant as described herein may comprise, or consist of SEQ ID NO: 75. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 75.

In some implementations, a PAL enzyme variant as described herein may comprise, or consist of SEQ ID NO: 76. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 76.

In some implementations, a PAL enzyme variant as described herein may comprise, or consist of SEQ ID NO: 77. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 77.

In some implementations, a PAL enzyme variant as described herein may comprise, or consist of SEQ ID NO: 78. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 78.

In some implementations, a PAL enzyme variant as described herein may comprise, or consist of SEQ ID NO: 79. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 79.

In some implementations, a PAL enzyme variant as described herein may comprise, or consist of SEQ ID NO: 80. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 80.

In some implementations, a PAL enzyme variant as described herein may comprise, or consist of SEQ ID NO: 81. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 81.

In some implementations, a PAL enzyme variant as described herein may comprise, or consist of SEQ ID NO: 82. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 82.

In some implementations, a PAL enzyme variant as described herein may comprise, or consist of SEQ ID NO: 83. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 83.

In some implementations, a PAL enzyme variant as described herein may comprise, or consist of SEQ ID NO: 84. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 84.

In some implementations, a PAL enzyme variant as described herein may comprise, or consist of SEQ ID NO: 85. In some implementations, a PAL enzyme variant as described herein has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the variant sequence of SEQ ID NO: 85.

For the purposes of the present disclosure, it is expected that the disclosed engineered PAL enzymes comprise at least one substitution mutation, deletion, and/or insertion that increases catalyzation of phenylalanine relative to a non-engineered enzyme comprising the same amino acid sequence but without the modification. In some implementations, a variant as described herein exhibits increased activity relative to wild-type PAL (SEQ ID NO: 1), such that its activity is at least about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 550%, about 600%, about 650%, about 700%, about 750%, about 800%, about 850%, about 900%, about 950%, about 1000%, about 1100%, about 1200%, about 1300%, about 1400%, about 1500%, about 1600%, about 1700%, about 1800%, about 1900%, about 2000%, about 2250%, about 2500%, about 2750%, about 3000%, about 3250%, about 3500%, about 3750%, about 4000%, about 4250%, about 4500%, about 4750%, about 5000%, about 10000%, about 100000%, about 1000000%, about 10000000%, or about 20000000% or more than that of the wild-type PAL, as determined by a method disclosed herein. Similarly, in some implementations, a variant as described herein exhibits increased activity relative to wild-type PAL (SEQ ID NO: 1), such that its activity is at least about 2-fold—e.g., at least about 4-fold, about 5-fold, about 10-fold, about 18-fold, about 20-fold, about 50-fold, about 100-fold, about 200-fold, about 1000-fold, about 5000-fold, about 10000-fold, about 20000-fold, about 50000-fold, about 100000-fold, about 200000-fold, about 500000-fold, or about 1000000-fold, or more, than that of the wild-type PAL, as determined by a method disclosed herein.

The enzyme variants of the present disclosure may include any of the substitutions described above and a deletion of the methionine residue at amino acid position 1.

### V. Pharmaceutical Compositions

The PAL enzyme variants disclosed herein can be formulated into pharmaceutical compositions suitable for administration to the target subject (i.e., a human or other mammal) via a predetermined route of administration, as discussed in more detail below.

Pharmaceutical compositions may include one or more variants as described herein and a pharmaceutically acceptable carrier or diluent.

The compositions may be formulated for intravenous, subcutaneous, intraperitoneal, intramuscular, oral, nasal, pulmonary, ocular, vaginal, or rectal administration. The composition may be formulated for administration by injection or infusion. The composition may be formulated for oral administration. The compositions may be formulated for intravenous, subcutaneous, intraperitoneal, or intramuscular administration, such as in a solution, suspension, emulsion, liposome formulation, etc. The pharmaceutical compositions can be formulated to be an immediate-release composition, sustained-release composition, delayed-release composition, etc., using techniques known in the art.

Preferably, the composition is formulated for liquid aerosol administration or dry powder aerosol administration. Inhaled protein therapeutics meet a growing interest for treatment of various disease. However, in aerosols, proteins and enzymes face stresses that may generate instabilities, such as physicochemical denaturation, aggregation, and loss of activity. Thus, it is important that the protein or enzyme used in liquid aerosol administration or dry powder aerosol administration is designed to better withstand said stresses.

Pharmaceutically acceptable carriers for various dosage forms are known in the art. For example, excipients, lubricants, binders, and disintegrants for solid preparations are known; solvent, solubilizing agents, suspending agents, isotonicity agents, buffers, and soothing agents for liquid preparations are known. The pharmaceutical compositions may include one or more additional components, such as one or more preservatives, antioxidants, colorants, sweetening/flavoring agents, absorbing agents, wetting agents, and the like.

In some embodiments, the PAL enzyme variant may be a long-acting variant that has been modified in order to extend its half-life in vivo (after administration). Various techniques are known in the art for extending the circulating half-life of peptides. For example, the variant may be conjugated to polyethylene glycol (PEG) or a similar polymer that prolongs half-life. Alternatively, the variant may be fused to an albumin-binding peptide, an albumin-binding protein domain, human serum albumin, or an inert polypeptide. Exemplary inert polypeptides that have been used to increase the circulating half-life of peptides include, but are not limited to, XTEN® (also known as recombinant PEG or “rPEG”), a homo-amino acid polymer (HAP; HAPylation), a proline-alanine serine polymer (PAS; PASylation), or an elastin-like peptide (ELP; ELPylation). As used herein, “fused to” includes genetic fusion, directly or through a linker, resulting in a single polypeptide containing multiple domains, unless otherwise specified.

### VI. Methods and Treatments

As noted above, the variants described herein are useful in methods of degrading phenylalanine in a mammalian subject in need thereof. The subject may be a human subject. The human subject may have PKU. Thus, the variants described herein may be useful in methods of treating PKU in a mammalian subject in need thereof.

The methods generally involve administering a therapeutically effective amount of a PAL enzyme variant as described herein (or a pharmaceutical composition comprising the same) to the subject. A therapeutically effective amount would be an amount that degrades phenylalanine to improve PKU or to negate the effects of phenylalanine accumulation in the subject. The method may include administering the enzyme variant itself to the subject. However, the method may include administering a nucleic acid encoding the PAL enzyme variant in a construct that expresses the variant in vivo. For example, the nucleic acid can be provided in a suitable vector, such as an adeno-associated virus (AAV) gene transfer vector. Other exemplary vectors that are suitable for use in such methods are known in the art. Exemplary vectors may include one or more enhancers (e.g., a cytomegalovirus (CMV) enhancer), promoters (e.g., chicken 3-actin promoter), and/or other elements enhancing the properties of the expression cassette. Methods of making suitable vectors and general methods of using expression vectors in vivo are known in the art.

A subject in need of treatment for PKU is a human subject who has increased levels of phenylalanine and decreased levels of tyrosine in their bloodstream. Such a subject may or may not exhibit brain damage, intellectual disability, seizures, tremors, neurological defects, behavioral defects, developmental defects, musty odor in breath or skin or urine, skin rashes, reduced melanin production, and hyperactivity.

A therapeutically effective amount of a PAL enzyme variant may be an amount effective to reduce the amount of phenylalanine in the bloodstream to a safe range of less than or equal to 300 μmol/L. A therapeutically effective amount of a PAL enzyme variant may be an amount effective to reduce the amount of phenylalanine in the bloodstream to a range observed with individuals without PKU which is less than or equal to 120 μmol/L.

The specific amount of a PAL enzyme that is administered may depend on one or more of the age and/or weight of the subject, the amount of phenylalanine the subject can degrade without treatment, and/or the specific mutation causing PKU. A variant may be administered at a dose of from about 0.01 to about 20 mg/kg, about 0.1 mg/kg to about 18 mg/kg, about 1 mg/kg to about 16 mg/kg, about 2 mg/kg to about 14 mg/kg, or about 5 mg/kg to about 10 mg/kg. In some embodiments, a variant is administered at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8/5 mg/kg, about 9 mg/kg, about 9.5 mg/kg, about 10 mg/kg, about 10.5 mg/kg, about 12 mg/kg, about 12.5 mg/kg, about 13 mg/kg, about 13.5 mg/kg, about 14 mg/kg, about 14.5 mg/kg, about 15 mg/kg, about 15.5 mg/kg, about 16 mg/kg, about 16.5 mg/kg, about 17 mg/kg, about 17.5 mg/kg, about 18 mg/kg, about 18.5 mg/kg, about 19 mg/kg, about 19.5 mg/kg, or about 20 mg/kg. A variant may be administered at a dose of about 0.5 mg, about 1 mg, about 2.5 mg, about 5 mg, about 10 mg, about 15 mg, about 20 mg, about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100, about 2150 mg, about 2200 mg, about 2250 mg, about 2300 mg, about 2350 mg, about 2400 mg, about 2450 mg, or about 2500 mg. When more than one variant is administered, the total amount of variants administered may be in accordance with the foregoing guidance.

The methods may be administered via a single dose of a PAL enzyme variant(s) (or composition comprising the same). The method may be administered via repeated doses, such as for a predetermined period of time until the symptoms or effects of PKU are reduced, ameliorated, or eliminated. Treatment may be repeated with additional doses of the variant(s) if signs/symptoms/effects persist. Treatment may be repeated for the duration of a subject's life.

The methods may include administering a PAL enzyme variant(s) (or composition including the same) three or more times a day, twice a day, or once a day. The methods may include administering a PAL enzyme variant(s) (or composition comprising the same) once every other day, three times a week, twice a week, once a week, once every other week, once every three weeks, once a month, or less frequently. The PAL enzyme variant may be a long-acting PAL enzyme variant as described above.

Treatment may continue for 1, 2, 3, 4, 5, 6, 7, 8, 9, 10, 11, 12, 13, 14, 15, 16, 17, 18, 19, 20, 21 or more days; 1, 2, 3, 4, 5, 6, 7, 8, 9, 10, 11, 12, 13, 14, 15, 16, 17, 18 or more weeks; or 1, 2, 3, 4, 5, 6, 7, 8, 9, 10, 11, 12 or more months; or 1, 2, 3 or more years or until the subject no longer exhibits symptoms of PKU.

The method may be a method of treating PKU in a subject with the disease. The method may be a method of degrading phenylalanine in a subject.

One skilled in the art will readily appreciate that the present disclosure is well adapted to carry out the objects and obtain the ends and advantages mentioned, as well as those inherent therein. Modifications therein and other uses will occur to those skilled in the art. These modifications are encompassed within the spirit of the disclosure. The following examples are given to illustrate the present invention. It should be understood, however, that the invention is not limited to the specific conditions or details of these examples.

### VII. Select Embodiments

The present disclosure is not to be limited to the particular embodiments described below. Rather, these select embodiments are intended as illustrative of certain aspects of the present technology.

Embodiment 1: A phenylalanine-degrading enzyme variant comprising an amino acid sequence that is a variant of the amino acid sequence of the wild-type phenylalanine ammonia lyase (PAL) enzyme set forth in SEQ ID NO: 1, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1.

Embodiment 2: The variant of embodiment 1, wherein the substitution increases the thermostability of the variant relative to the wild-type PAL enzyme.

Embodiment 3: The variant of embodiment 2, wherein the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C.

Embodiment 4: The variant of embodiment 2, wherein the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C.

Embodiment 5: The variant of embodiment 3 or 4, wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

Embodiment 6: The variant of any one of embodiments 1-5, wherein the substitution increases the residual PAL activity following nebulization relative to the wild-type PAL enzyme.

Embodiment 7: The variant of embodiment 6, wherein the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge.

Embodiment 8: The variant of embodiment 6 or 7, wherein the amount of PAL activity is determined by measuring the amount of cinnamic acid produced via liquid chromatography with UV detection following a 30-minute incubation of 21 μg of nebulization challenged enzyme with 25 μL of 80 mM phenylalanine.

Embodiment 9: The variant of embodiment 7 or 8, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

Embodiment 10: The variant of any one of embodiments 1-9, wherein the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

Embodiment 11: The variant of any one of embodiments 1-10, wherein the variant sequence comprises an amino acid sequence selected from any one of SEQ ID NOs: 2-41.

Embodiment 12: The variant of any one of embodiments 1-10, wherein the variant sequence comprises at least two substitutions at two or more amino acid positions selected from 25, 30, 56, 64, 70, 96, 104, 247, 285, 457, 461, 483, 503, and 521 of SEQ ID NO: 1.

Embodiment 13: The variant of any one of embodiments 1-10, wherein the variant sequence comprises at least three substitutions at three or more amino acid positions selected from 25, 30, 56, 64, 70, 96, 104, 247, 285, 457, 461, 483, 503, and 521 of SEQ ID NO: 1.

Embodiment 14: The variant of embodiment 13, wherein the variant sequence comprises at least three substitutions selected from N25E, N30E, I56V, C64V, A70S, Q96E, L104M, M247L, I285L, Q457M, S461A, G483A, C503T, and Q521V.

Embodiment 15: The variant of embodiment 13 or 14, wherein the variant sequence comprises an amino acid sequence selected from any one of SEQ ID NOs: 42-47.

Embodiment 16: The variant of any one of embodiments 1-10, wherein the variant sequence comprises at least four substitutions at four or more amino acid positions selected from 25, 30, 64, 70, 96, 104, 247, 285, 457, 461, 483, 503, and 521 of SEQ ID NO: 1.

Embodiment 17: The variant of embodiment 16, wherein the variant sequence comprises at least four substitutions selected from N25E, N30E, C64V, A70S, Q96E, L104M, M247L, I285L, Q457M, S461A, G483A, C503T, and Q521V.

Embodiment 18: The variant of embodiment 16 or 17, wherein the variant sequence comprises an amino acid sequence selected from any one of SEQ ID NOs: 48-52.

Embodiment 19: The variant of any one of embodiments 1-10, wherein the variant sequence comprises at least five substitutions at five or more amino acid positions selected from 30, 64, 70, 96, 104, 483, 503, and 521 of SEQ ID NO: 1.

Embodiment 20: The variant of embodiment 19, wherein the variant sequence comprises at least five substitutions selected from N30E, C64V, A70S, Q96E, L104M, G483A, C503T, and Q521V.

Embodiment 21: The variant of embodiment 19 or 20, wherein the variant sequence comprises an amino acid sequence selected from any one of SEQ ID NOs: 53-58.

Embodiment 22: The variant of any one of embodiments 1-10, wherein the variant sequence comprises at least six substitutions at six or more amino acid positions selected from 30, 64, 70, 96, 104, 483, 503, and 521 of SEQ ID NO: 1.

Embodiment 23: The variant of embodiment 22, wherein the variant sequence comprises at least six substitutions selected from N30E, C64V, A70S, Q96E, L104M, G483A, C503T, and Q521V.

Embodiment 24: The variant of embodiment 22 or 23, wherein the variant sequence comprises an amino acid sequence selected from any one of SEQ ID NOs: 59-61.

Embodiment 25: The variant of any one of embodiments 1-10, wherein the variant sequence comprises at least eight substitutions at eight or more amino acid positions selected from 30, 64, 70, 96, 104, 483, 503, and 521 of SEQ ID NO: 1.

Embodiment 26: The variant of embodiment 25, wherein the variant sequence comprises at least eight substitutions selected from N30E, C64V, A70S, Q96E, L104M, G483A, C503T, and Q521V.

Embodiment 27: The variant of embodiment 25 or 26, wherein the variant sequence comprises an amino acid sequence of SEQ ID NO: 62.

Embodiment 28: The variants of any of embodiments 1-27, wherein the variant sequence comprises a deletion of a methionine residue at amino acid position 1 of SEQ ID NO: 1.

Embodiment 29: The variant of any one of embodiments 1-28 for use in treating phenylketonuria (PKU).

Embodiment 30: Use of a variant of any one of embodiments 1-28 in the manufacture of a medicament for treating phenylketonuria (PKU).

Embodiment 31: A pharmaceutical composition, comprising: (a) a phenylalanine-degrading enzyme variant according to any one of embodiments 1-29, and (b) a pharmaceutically acceptable carrier or excipient.

Embodiment 32: The pharmaceutical composition of embodiment 31, wherein the pharmaceutical composition is formulated for aerosol administration.

Embodiment 33: The pharmaceutical composition of embodiment 32, wherein the aerosol administration is selected from liquid aerosol administration or dry powder aerosol administration.

Embodiment 34: A pharmaceutical composition, comprising: a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme; and a pharmaceutically acceptable carrier.

Embodiment 35: The pharmaceutical composition of embodiment 34, wherein the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C.

Embodiment 36: The pharmaceutical composition of embodiment 34, wherein the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C.

Embodiment 37: The pharmaceutical composition of embodiment 35 or 36, wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

Embodiment 38: The pharmaceutical composition of embodiment 34, wherein the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge.

Embodiment 39: The pharmaceutical composition of embodiment 38, wherein the amount of PAL activity is determined by measuring the amount of cinnamic acid produced via liquid chromatography with UV detection following a 30-minute incubation of 21 μg of nebulization challenged enzyme with 25 μL of 80 mM phenylalanine.

Embodiment 40: The pharmaceutical composition of embodiment 38 or 39, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

Embodiment 41: The pharmaceutical composition of embodiment 34-40, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

Embodiment 42: The pharmaceutical composition of embodiment 34-41, wherein the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

Embodiment 43: The pharmaceutical composition of any one of embodiments 34-42, wherein the variant sequence comprises an amino acid sequence selected from any one of SEQ ID NOs: 2-62.

Embodiment 44: The pharmaceutical composition of any one of embodiments 34-43, wherein the pharmaceutical composition is formulated for aerosol administration.

Embodiment 45: The pharmaceutical composition of embodiment 44, wherein the aerosol administration is selected from liquid aerosol administration or dry powder aerosol administration.

Embodiment 46: The pharmaceutical composition of any one of embodiments 31-45 for use in treating phenylketonuria (PKU).

Embodiment 47: Use of a pharmaceutical composition of any one of embodiments 31-45 in the manufacture of a medicament for treating phenylketonuria (PKU).

Embodiment 48: A method of treating a subject with PKU, the method comprising administering to the subject a variant according to any one of embodiments 1-29 or a pharmaceutical composition according to any one of embodiments 31-45.

Embodiment 49: The method of embodiment 48, wherein the variant or the pharmaceutical composition is administered as a liquid aerosol or a dry powder aerosol.

Embodiment 50: A method of treating a subject with PKU, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.

Embodiment 51: The method of embodiment 50, wherein the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C.

Embodiment 52: The method of embodiment 50, wherein the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C.

Embodiment 53: The method of embodiment 51 or 52, wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

Embodiment 54: The method of embodiment 50, wherein the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge.

Embodiment 55: The method of embodiment 54, wherein the amount of PAL activity is determined by measuring the amount of cinnamic acid produced via liquid chromatography with UV detection following a 30-minute incubation of 21 μg of nebulization challenged enzyme with 25 μL of 80 mM phenylalanine.

Embodiment 56: The method of embodiment 54 or 55, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

Embodiment 57: The method of embodiment 50-56, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

Embodiment 58: The method of embodiment 50-57, wherein the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

Embodiment 59: The method of any one of embodiments 50-58, wherein the variant sequence comprises two to fourteen substitutions at two to fourteen amino acid positions selected from 25, 30, 56, 64, 70, 96, 104, 247, 285, 457, 461, 483, 503, and 521 of SEQ ID NO: 1.

Embodiment 60: The method of embodiment 59, wherein the two to fourteen substitutions are selected from N25E, N30E, I56V, C64V, A70S, Q96E, L104M, M247L, I285L, Q457M, S461A, G483A, C503T, and Q521V.

Embodiment 61: The method of embodiment 59 or 60, wherein the variant sequence comprises 2, 3, 4, 5, 6, 7, 8, 9, 10, 11, 12, 13, or 14 substitutions.

Embodiment 62: The method of any one of embodiments 50-61, wherein the variant sequence comprises an amino acid sequence selected from SEQ ID NO: 2-62.

Embodiment 63: The method of any one of embodiments 50-62, wherein the enzyme variant is formulated for aerosol administration.

Embodiment 64: The method of embodiment 63, wherein the aerosol administration is selected from liquid aerosol administration or dry powder aerosol administration.

Embodiment 65: A method of degrading phenylalanine in a subject, the method comprising administering to the subject a variant according to any one of embodiments Error!Reference source not found.-29 or a pharmaceutical composition according to any one of embodiments 31-45.

Embodiment 66: The method of embodiment 65, wherein the variant or the pharmaceutical composition is formulated for aerosol administration.

Embodiment 67: The method of embodiment 66, wherein the aerosol administration is selected from liquid aerosol administration or dry powder aerosol administration.

Embodiment 68: The method of any one of embodiments 65-67, wherein the subject has PKU.

Embodiment 69: A method of degrading phenylalanine in a subject, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.

Embodiment 70: The method of embodiment 69, wherein the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C.

Embodiment 71: The method of embodiment 69, wherein the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C.

Embodiment 72: The method of embodiment 70 or 71, wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

Embodiment 73: The method of embodiment 69, wherein the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge.

Embodiment 74: The method of embodiment 73, wherein the amount of PAL activity is determined by measuring the amount of cinnamic acid produced via liquid chromatography with UV detection following a 30-minute incubation of 21 μg of nebulization challenged enzyme with 25 μL of 80 mM phenylalanine.

Embodiment 75: The method of embodiment 73 or 74, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

Embodiment 76: The method of any one of embodiments 69-75, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO: 1.

Embodiment 77: The method of any one of embodiments 69-76, wherein the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

Embodiment 78: The method of any one of embodiments 69-77, wherein the variant sequence comprises two to fourteen substitutions at two to fourteen amino acid positions selected from 25, 30, 56, 64, 70, 96, 104, 247, 285, 457, 461, 483, 503, and 521 of SEQ ID NO: 1.

Embodiment 79: The method of embodiment 78, wherein the two to fourteen substitutions are selected from N25E, N30E, I56V, C64V, A70S, Q96E, L104M, M247L, I285L, Q457M, S461A, G483A, C503T, and Q521V.

Embodiment 80: The method of embodiment 78 or 79, wherein the variant sequence comprises 2, 3, 4, 5, 6, 7, 8, 9, 10, 11, 12, 13, or 14 substitutions.

Embodiment 81: The method of any one of embodiments 69-80, wherein the variant sequence comprises an amino acid sequence selected from SEQ ID NO: 2-62.

Embodiment 82: The method of any one of embodiments 69-81, wherein the enzyme variant is formulated for aerosol administration.

Embodiment 83: The method of embodiment 82, wherein the aerosol administration is selected from liquid aerosol administration or dry powder aerosol administration.

Embodiment 84: The method of any one of embodiments 69-83, wherein the subject has phenylketonuria (PKU).

### EXAMPLES

The present technology is further illustrated by the following Examples, which should not be construed as limiting in any way.

### Example 1

Proteins tend to misfold and aggregate during delivery to and occupancy in the lungs. Literature and experts believe this misfolding and aggregation is caused by mechanical stress induced during aerosolization and protein migration at the air-liquid interfaces present in both aerosol droplets and mucus within the lungs. Ultimately, protein drugs delivered to the lungs must stay properly folded during delivery and after deposition. Thus, development of hyper-folded protein variants could enable a competitive advantage for both goals.

Metabolites diffuse between the lungs and the bloodstream. This interaction may be exploited to treat PKU by depositing enzymes in the lungs and depleting specific metabolites in the blood. Presumably, it would be possible to deposit an enzyme acting on specific blood metabolites in the lungs and then metabolize target molecules that freely diffuse between the lungs and blood. This hypothesis does not require the enzyme to translocate across the lung membrane and into the bloodstream. It, instead, depends upon the metabolites translocating from the bloodstream and into the lungs and upon the enzymes remaining active in the lungs.

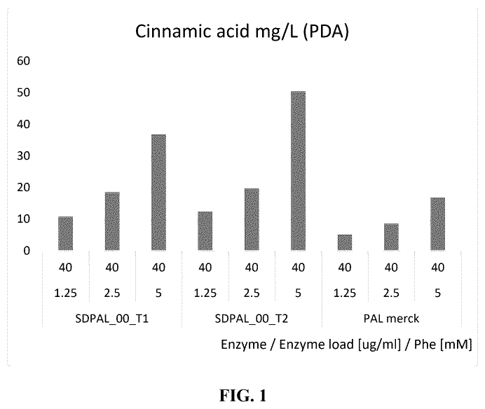

Phenylalanine ammonia lyase (PAL) from A. variabilis was selected as the enzyme used to generate variants as the enzyme did not require a co-factor for proper activity. Two wild-type PAL enzymes was expressed and tested against a commercially available PAL enzyme from Rhodotorula glutinis. The wild-type A. variabilis enzymes and R. glutinis enzyme were tested for PAL activity. 40 mM of phenylalanine was combined with an enzyme load of 1.25, 2.5, and 5 μg/mL for each enzyme. Activity was measured by detecting the amount of cinnamic acid released. All three tested enzymes were active, with the generated wild-type A. variabilis PAL enzymes exhibiting increased activity as compared to the commercially available recombinant R. glutinis PAL enzyme (FIG. 1).

A sequential process was applied to identify and qualify PAL mutants. Two rounds of selection were used to identify candidate variants. The first round focused on single amino acid changes, while the second round focused on stacking the best single variants to generate variants with more than one amino acid change.

40 alleles of A. variabilis PAL were selected using a convolutional neural network (CNN) that was run on the PAL structure from A. variabilis (Table 2). The top 40 single-point mutation variants were selected based on the following criteria: 1) data were arranged by their ground truth probability (pGT) or accuracy of the training set's classification for the supervised learning techniques from low to high; 2) data points with pGT less than 0.1 were selected; 3) the selected data points were sorted for a high probability difference (pMut−pGT); 4) the top 60 mutations were selected and visually inspected on the PAL structure for the suitability of the mutated residue at a given position; 5) any mutations consisting of conserved residues in the active site were removed; and 6) the final top 40 mutations were selected. These models provided fitness probabilities for both ground truth amino acids (Prob GT, pGT) and the remaining 19 amino acids as mutations (Prob Mut, pMut) at each position.

TABLE 2
List of PAL Mutants Generated in the First Round of Identification

| Name | Mutation | SEQ ID NO |
|---|---|---|
| SDPAL_00 | WT PAL | SEQ ID NO: 1 |
| SDPAL-01 | N25E | SEQ ID NO: 2 |
| SDPAL-02 | N30E | SEQ ID NO: 3 |
| SDPAL-03 | N44E | SEQ ID NO: 4 |
| SDPAL-04 | T46V | SEQ ID NO: 5 |
| SDPAL-05 | I56V | SEQ ID NO: 6 |
| SDPAL-06 | I56T | SEQ ID NO: 63 |
| SDPAL-07 | C64V | SEQ ID NO: 7 |
| SDPAL-08 | A70S | SEQ ID NO: 64 |
| SDPAL-09 | Q96E | SEQ ID NO: 8 |
| SDPAL-10 | L104M | SEQ ID NO: 9 |
| SDPAL-11 | T110V | SEQ ID NO: 10 |
| SDPAL-12 | K115Y | SEQ ID NO: 11 |
| SDPAL-13 | Y160K | SEQ ID NO: 65 |
| SDPAL-14 | F188Y | SEQ ID NO: 66 |
| SDPAL-15 | N234L | SEQ ID NO: 12 |
| SDPAL-16 | M247L | SEQ ID NO: 13 |
| SDPAL-17 | D253H | SEQ ID NO: 14 |
| SDPAL-18 | D253F | SEQ ID NO: 67 |
| SDPAL-19 | I285L | SEQ ID NO: 15 |
| SDPAL-20 | L316I | SEQ ID NO: 16 |
| SDPAL-21 | N347D | SEQ ID NO: 68 |
| SDPAL-22 | Q366M | SEQ ID NO: 69 |
| SDPAL-23 | Q389N | SEQ ID NO: 17 |
| SDPAL-24 | M416Q | SEQ ID NO: 18 |
| SDPAL-25 | Q422E | SEQ ID NO: 70 |
| SDPAL-26 | C424T | SEQ ID NO: 19 |
| SDPAL-27 | N437E | SEQ ID NO: 20 |
| SDPAL-28 | A440S | SEQ ID NO: 21 |
| SDPAL-29 | E448D | SEQ ID NO: 22 |
| SDPAL-30 | Q449E | SEQ ID NO: 23 |
| SDPAL-31 | Q457M | SEQ ID NO: 24 |
| SDPAL-32 | S461A | SEQ ID NO: 25 |
| SDPAL-33 | Q473K | SEQ ID NO: 71 |
| SDPAL-34 | N474V | SEQ ID NO: 26 |
| SDPAL-35 | G483A | SEQ ID NO: 27 |
| SDPAL-36 | G483S | SEQ ID NO: 72 |
| SDPAL-37 | C503T | SEQ ID NO: 28 |
| SDPAL-38 | Q521V | SEQ ID NO: 29 |
| SDPAL-39 | Q521E | SEQ ID NO: 73 |
| SDPAL-40 | Y529F | SEQ ID NO: 74 |
| SDPAL-41 | N534D | SEQ ID NO: 30 |

The next step was to test the top 40 enzyme variants for PAL activity in vitro. The variants were producing using the in vitro transcription and translation (IVTT) platform from Tierra. The variants were tagged to facilitate purification from the protein synthesis IVTT reaction solution.

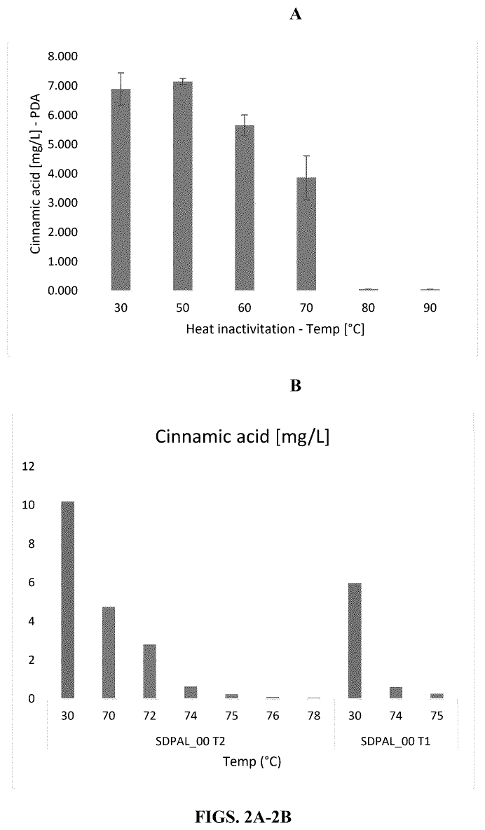

The purified proteins were then subjected to a thermal challenge assay to identify resilient, hyper-folded PAL variants with the goal to find variants which retain strong PAL activity after thermal stress relative to the wild-type PAL enzyme. To design an effective thermal challenge assay, the wild-type enzyme was exposed to a range of temperatures for ten minutes and then tested for their ability to process phenylalanine to produce cinnamic acid. The temperatures tested were 30° C., 50° C., 60° C., 70° C., 80° C., and 90° C. The purified wild-type PAL enzyme exhibited significant cinnamic acid production from 30° C. to 70° C. but did not exhibit much activity at either 80° C. or 90° C. (FIG. 2A). To determine the optimal temperature where the wild-type PAL enzyme lost most enzymatic activity, a smaller window of temperatures of 70° C. to 78° C. were tested in 2° C. increments for ten minutes (FIG. 2B). It was determined that the temperature where wild-type PAL enzyme began exhibiting dramatic decreases in activity was 74° C. Therefore, this temperature was chosen for use in the thermal challenge assay for the enzyme variants.

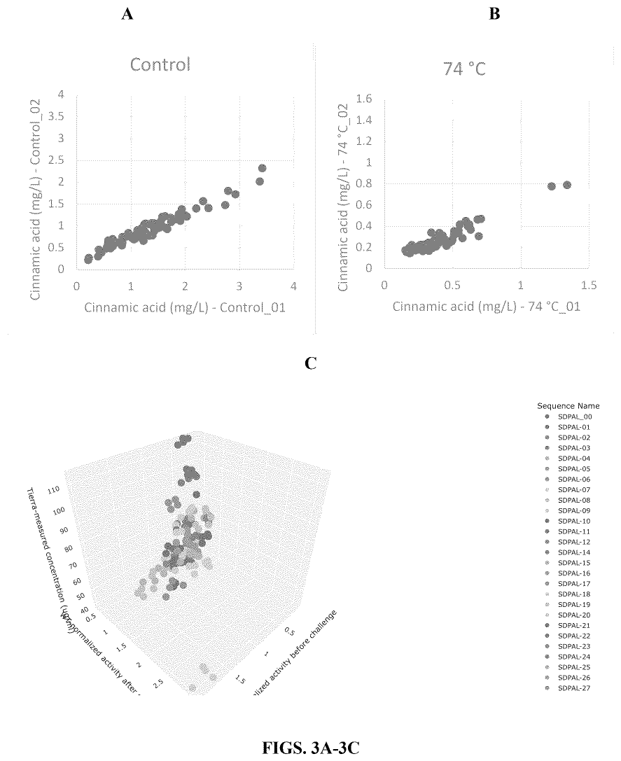

The 40 identified enzyme variants were tested for PAL activity after a ten-minute thermal challenge at 74° C. (FIGS. 3A-3C). It was observed that around 60% of the enzyme variants exhibited improved retained activity after thermal challenge and that around 20% of the variants exhibited improved catalytic turnover prior to thermal challenge. In addition, around 20% of the variants were shown to be more active after a thermal challenge that the wild-type PAL enzyme prior to thermal challenge. Thus, this study revealed that some PAL enzyme variants are more resilient compared to the wild-type PAL enzyme (Table 3).

TABLE 3
Activity Before and After Thermal Challenge for Single PAL Variants
Single variants with 2 ug/ml enzyme conc.:

| | | Cinnamic | Cinnamic | Residual |
|---|---|---|---|---|
| | | acid [mg/L] | acid [mg/L] | activity |
| Name | Mutation | After | Before | (%) |
| SDPAL_09 | Q96E | 0.88 | 2.129 | 41.34% |
| SDPAL_07 | C64V | 0.682 | 1.864 | 36.57% |
| SDPAL_02 | N30E | 0.454 | 0.805 | 56.34% |
| SDPAL_35 | G483A | 0.426 | 1.949 | 21.86% |
| SDPAL_38 | Q521V | 0.441 | 1.636 | 26.93% |
| SDPAL_10 | L104M | 0.873 | 1.552 | 56.25% |
| SDPAL_19 | I285L | 0.363 | 0.678 | 53.51% |
| SDPAL_37 | C503T | 0.482 | 2.188 | 22.01% |
| SDPAL_00 | WT | 0.351 | 1.452 | 24.18% |

In addition to the 40 initially identified enzyme variants, 18 additional single amino acid variants and 25 stacked variants, which include between three and eight amino acid changes, were designed (Table 4). The stacked variants were designed by: 1) analyzing the different activity data, including the PAL enzymatic activity before and after thermal challenge, the fraction of retained activity, and variant ratio relative to wild-type ratio to identify the top single-point variants; 2) selecting single-point variants with activity values higher than a threshold value with respect to wild-type activity; 3) combining and sorting the selected single-point variants based on their frequency of occurrence in multiple activity data sets; 4) composing stacked variants with the top five single-point variants with higher identified frequencies; and 5) creating stacked variants with a combination of three to eight single-point variants, with no two mutations closer than 20 angstroms to one another.

TABLE 4
List of PAL Mutants Generated in the
Second Round of Identification

| Name | Mutation | SEQ ID NO |
|---|---|---|
| Wild-type | WT PAL | SEQ ID NO: 1 |
| RDPAL_1 | A70S, Q96E, G483A | SEQ ID NO: 82 |
| RDPAL_2 | Q96E, G483A, C503T | SEQ ID NO: 42 |
| RDPAL_3 | A70S, G483A, C503T | SEQ ID NO: 83 |
| RDPAL_4 | A70S, Q96E, G483A, C503T | SEQ ID NO: 84 |
| RDPAL_5 | A70S, Q96E, G483A, C503T, N30E | SEQ ID NO: 53 |
| RDPAL_6 | A70S, Q96E, G483A, C503T, L104M | SEQ ID NO: 54 |
| RDPAL_7 | A70S, Q96E, G483A, C503T, I285L, | SEQ ID NO: 59 |
| | Q521V | |
| RDPAL_8 | N30E, C64V. A70S, Q96E, L104M, | SEQ ID NO: 62 |
| | G483A, C503T, Q521V | |
| RDPAL_9 | Q96E, C64V, N30E, G483A, C503T, | SEQ ID NO: 60 |
| | Q521V | |
| RDPAL_10 | L104M, C64V, N30E, G483A, C503T, | SEQ ID NO: 61 |
| | Q521V | |
| RDPAL_11 | L104M, C64V, N30E, G483A, C503T | SEQ ID NO: 55 |
| RDPAL_12 | Q96E, C64V, N30E, G483A, C503T | SEQ ID NO: 56 |
| RDPAL_13 | L104M, C64V, N30E, G483A, Q521V | SEQ ID NO: 57 |
| RDPAL_14 | Q96E, C64V, N30E, G483A, Q521V | SEQ ID NO: 58 |
| RDPAL_15 | I56V, M247L, Q457M | SEQ ID NO: 85 |
| RDPAL_16 | I56V, Q457M, Q521V | SEQ ID NO: 43 |
| RDPAL_17 | I56V, S461A, Q521V | SEQ ID NO: 44 |
| RDPAL_18 | N25E, C64V, M247L, Q457M | SEQ ID NO: 48 |
| RDPAL_19 | N25E, C64V, M247L, S461A | SEQ ID NO: 49 |
| RDPAL_20 | N30E, Q96E, I285L | SEQ ID NO: 45 |
| RDPAL_21 | N30E, Q96E, C503T | SEQ ID NO: 46 |
| RDPAL_22 | C64V, L104M, C503T | SEQ ID NO: 47 |
| RDPAL_23 | N30E, Q96E, I285L, C503T | SEQ ID NO: 50 |
| RDPAL_24 | C64V, L104M, I285L, Q521V | SEQ ID NO: 51 |
| RDPAL_25 | N30E, L104M, I285L, Q521V | SEQ ID NO: 52 |
| SDPAL-42 | I27V | SEQ ID NO: 31 |
| SDPAL-43 | Q31R | SEQ ID NO: 32 |
| SDPAL-44 | R43Y | SEQ ID NO: 75 |
| SDPAL-45 | L47S | SEQ ID NO: 33 |
| SDPAL-46 | E72K | SEQ ID NO: 76 |
| SDPAL-47 | I92D | SEQ ID NO: 77 |
| SDPAL-48 | K115L | SEQ ID NO: 34 |
| SDPAL-49 | L118K | SEQ ID NO: 35 |
| SDPAL-50 | R134K | SEQ ID NO: 36 |
| SDPAL-51 | S209A | SEQ ID NO: 37 |
| SDPAL-52 | K216R | SEQ ID NO: 78 |
| SDPAL-53 | N223E | SEQ ID NO: 79 |
| SDPAL-54 | A246S | SEQ ID NO: 38 |
| SDPAL-55 | D282A | SEQ ID NO: 39 |
| SDPAL-56 | I310V | SEQ ID NO: 40 |
| SDPAL-57 | Q311E | SEQ ID NO: 80 |
| SDPAL-58 | R313K | SEQ ID NO: 41 |
| SDPAL-59 | Q321E | SEQ ID NO: 81 |

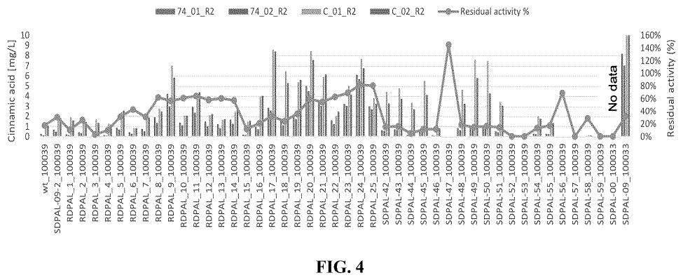

These variants were tested using the same thermal challenge assay (FIG. 4). Two of the stacked enzyme variants retained 80% of PAL activity after the thermal challenge, exhibited three times as much substrate turnover that wild-type PAL prior to thermal challenge, and showed almost no insoluble aggregated species during expression in E. coli, while more than half of the wild-type protein was present in inclusion bodies in E. coli.

The thermal challenge assay demonstrated that some of the enzyme variants were more stable than the wild-type PAL enzyme (Table 5). This increased stability suggests that these variants would remain properly folded and therefore active upon application of a variety of mechanical stress factors beyond just thermal challenge.

TABLE 5
Activity Data Before and After Thermal
Challenge for Stacked PAL Variants
Stacked variants with 8 ug/ml enzyme conc.:

| | | Cinnamic | Cinnamic | |
|---|---|---|---|---|
| | | acid | acid | Residual |
| | | [mg/L] | [mg/L] | activity |
| Name | Mutation | After | Before | (%) |
| SDPAL_00 | WT | 0.2 | 1.139 | 18% |
| RDPAL_9 | Q96E, C64V, | 3.606 | 6.421 | 56% |
| | N30E, G483A, | | | |
| | C503T, Q521V | | | |
| RDPAL_24 | C64V, L104M, | 5.881 | 7.207 | 82% |
| | I285L, Q521V | | | |
| RDPAL_20 | N30E, Q96E, | 4.742 | 7.993 | 59% |
| | I285L | | | |
| RDPAL_11 | L104M, C64V, | 2.629 | 4.104 | 64% |
| | N30E, G483A, | | | |
| | C503T | | | |

One relevant factor is residual PAL activity following nebulization. To test if the enzyme variants that retain increased residual activity following thermal challenge also exhibit increased residual activity following nebulization, four stacked variants were selected. The enzyme variants were tagged at the C-terminus by the C-tag, consisting of the E-P-E-A peptide fused directly to the protein. The tagged enzyme variants were expressed in E. coli and purified via elusion from an affinity matrix recognizing the tag.

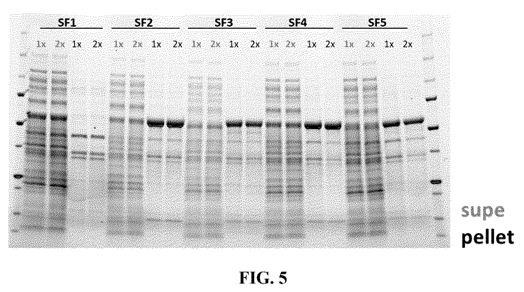

These variants were made in milligram quantities in a 5L fermenter. The enzyme variants and wild-type control were produced in standard lactose-containing autoinduction media. Fermentation was conducted at 24° C. After 16 hours, when CO₂ production dropped, the media was collected and centrifuged to pellet the cells. The cells were then resuspended in 20 mM Tris buffer (pH 7.4) and lysed by passing the cells through a cell homogenizer twice. The cell lysate was clarified by centrifugation, and the supernatant was applied to the C-tag affinity column. After binding, the column was washed with 3% Buffer B (20 mM Tris+2 M MgCl₂) and eluted in Buffer B. The elute was buffer exchanged to 1 M NaCl, concentrated to 60-70 g/L, aliquoted and stored at −20° C. The samples were run on an SDS-PAGE gel. (FIG. 5). Most of the enzyme variants were expressed intracellularly. The enzyme was enriched during C-tag based affinity purification and became the dominant band after elution. The soluble fraction of the cell lysate for the samples showed significant PAL activity.

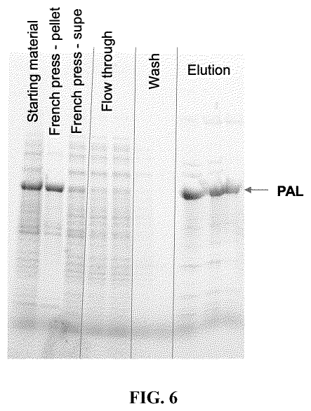

It appeared that the RDPAL_9 variant was more soluble than the other variants, as the other variants were mostly detected in the pellet fraction as opposed to the supernatant fraction in the SDS-PAGE analysis, suggesting the enzyme was localized in inclusion bodies. (FIGS. 5-6).

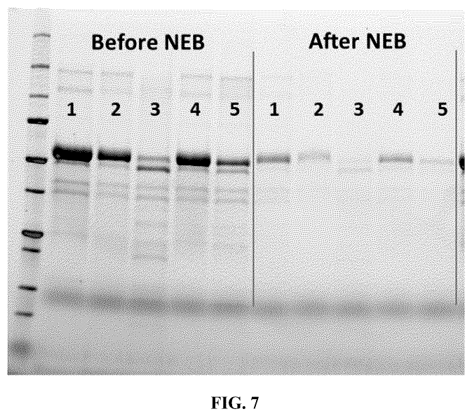

The purified enzyme variants and wild-type PAL enzyme were nebulized and then assessed for PAL activity. The enzyme samples were loaded into the nebulizer, and vacuum was applied to pull air into the nebulizer to form a mist with the samples. The mist was captured in an ice trap. 3 mL of protein volume was applied to the nebulizer, with the protein concentration varying from 0.5 to 3 g/L in 2 M MgCl₂ or 1 M NaCl. The amount of protein recovered and the activity of the recovered proteins were assessed. The variants and wild-type protein retained activity following nebulization, but there was protein lost during the nebulization process. (FIG. 7). On average, 1.5 to 2 mL of the liquid enzyme samples were recovered. Three of the enzyme variants exhibited superior residual PAL activity following nebulization compared to the wild-type PAL enzyme, demonstrating that the proteins are hyper-folded and more resilient to stressors (Table 6).

TABLE 6
Specific Activity of PAL Variants Before and After Nebulization

| | Specific Activity | | |
|---|---|---|---|
| | (μg Cinnamic Acid/1 μg PAL/1 hr) | | Residual |
| | Before | After | Specific |
| Name | Nebulization | Nebulization | Activity |
| RDPAL_9 | 20.9 | 15.3 | 73% |
| RDPAL_24 | 18.5 | 12.4 | 67% |
| RDPAL_20 | 9.5 | 2.6 | 28% |
| RDPAL_11 | 16.7 | 12.4 | 74% |
| Wild-Type | 19.8 | 8.2 | 41% |

### Example 2: Materials and Methods

PAL Activity Measurement: The enzyme phenylalanine ammonia lyase (PAL) catalyzes the conversion of L-phenylalanine to ammonia and trans-cinnamic acid. The assay utilized a 100 mM Tris-HCl, pH 7.0 buffer solution and a substrate stock solution of 80 mM phenylalanine in the buffer solution. The stop solution used was absolute ethanol. The control enzyme used was 0.7 to 1 μg/μL PAL. [00291]25 μL of the PAL sample or control enzyme was mixed with 25 μL of the substrate stock solution, resulting in a final phenylalanine concentration of 40 mM. The mixture was incubated for 30 minutes at 30° C. The reaction was stopped by adding 150 μL of the stop solution to the mixture. The sample was filtered through a 0.2 μm filter plate. The levels of cinnamic acid and phenylalanine were detected via high-performance liquid chromatography (HPLC) with UV detection. Typically, 21 μg of PAL was tested.

PAL Thermal Challenge Assay: The enzyme samples were prepared with 100 mM Tris HCl, pH 7.0, to generate a final volume of 25 μL with the same concentration of between 0.7 to 1 μg/μL. The enzyme samples were tested in duplicate and treated thermal challenge of 74° C. for 10 minutes.

Following thermal challenge, the activity of the enzyme variants were treated. The 25 μL treated samples were combined with 25 μL of 80 mM phenylalanine in 100 mM Tris-HCl, pH 7.0. The final concentration of the solution was 40 mM phenylalanine in 100 mM Tris-HCl and 21 μg thermal challenged PAL enzyme. The samples were held at 30° C. for 30 minutes and then quenched with 150 μL of an ethanol dilution four times. The levels of cinnamic acid and phenylalanine were detected via high-performance liquid chromatography (HPLC) with UV detection.

Nebulization Challenge Assay: The enzyme variants with C-tags were expressed in E. coli. The enzyme variants were purified using the C-tag according to the C-tag resin manufacturer recommendations (ThermoFisher CaptureSelect™ C-tag XL Affinity Matrix).

3 mL of enzyme volume was applied to the nebulizer. The enzyme concentration varies from 0.5 to 3 g/L in 2 M MgCl₂ or 1 NaCl.

Two models of nebulizers were used: the Philips Inno Spire Go and the Portable Mesh Nebulizer Model YM-3R9. The enzyme samples were loaded into the nebulizer, vacuum was applied to pull air into the nebulizer and form a mist using the liquid enzyme sample. The mist was captured in an ice trap, and the liquid enzyme samples were recovered. On average, a volume of 1.5 to 2 mL of the sample was recovered.

Two criteria after nebulization challenge were examined to determine the best variants: the amount of protein recovered and the activity of the recovered proteins.

PAL activity of the starting solution and the recovered solution was determined by measuring the levels of cinnamic acid and phenylalanine, which were detected via high-performance liquid chromatography (HPLC) with UV detection.

LC-PDA Method for Phenylalanine and Cinnamic Acid Determination: Analysis of phenylalanine substrate and cinnamic acid product for the PAL activity assays was performed using an HPLC method. A Waters Acquity Premier BEH Shield RP18 1.7 μm UHPLC column (2.1×50 mm, part number 186009500) was used. Phenylalanine was detected using 260 nm wavelength, while cinnamic acid was detected using 280 nm wavelength and a photo-diode array (PDA) detector. Millipore quality water with 0.1% trifluoroacetic acid (TFA) (A) and acetonitrile with 0.1% trifluoroacetic acid (TVA) (B) were used as mobile phases at a flow rate of 300 μL/minute. The injection volume was 2 μL. The column temperature was kept at 35° C. The initial conditions were 0% B for 2 minutes, raised to 20% B until minute 3, 30% B until minute 5, and 60% B until minute 6 and held at 60% B for 0.1 minutes and held at 0% 1.5 minutes for re-equilibration of the system. The quantitation limit for phenylalanine and cinnamic acid were found to be 10 mg/L and 0.025 mg/L, respectively.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

What is claimed is:

1. A phenylalanine-degrading enzyme variant comprising an amino acid sequence that is a variant of the amino acid sequence of the wild-type phenylalanine ammonia lyase (PAL) enzyme set forth in SEQ ID NO: 1, wherein the variant sequence comprises at least one substitution at one or more of positions selected from 25, 27, 30, 31, 44, 46, 47, 56, 64, 70, 96, 104, 110, 115, 118, 134, 209, 234, 246, 247, 253, 282, 285, 310, 313, 316, 389, 416, 424, 437, 440, 448, 449, 457, 461, 474, 483, 503, 521, and 534 of SEQ ID NO:1, wherein the substitution increases the thermostability of the variant relative to the wild-type PAL enzyme.

2. The variant of claim 1, wherein the increased thermostability of the variant relative to the wild-type PAL enzyme is an increase of at least 10% of residual PAL activity following a thermal challenge, wherein residual PAL activity for each enzyme is calculated by dividing the amount of PAL activity following thermal challenge by the amount of PAL activity prior to thermal challenge and wherein the thermal challenge comprises a 10-minute incubation at 74° C., wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

3. The variant of claim 1, wherein the increased thermostability of the variant is an increase in average PAL activity of at least 0.05 μg/mL of cinnamic acid produced relative to wild-type PAL enzyme following thermal challenge, wherein the thermal challenge comprises a 10-minute incubation at 74° C., wherein the amount of PAL activity is determined by measuring an amount of cinnamic acid produced via liquid chromatography with UV detection following a 30-minute incubation of 25 μL of 0.7 to 1 μg/μL unchallenged and thermal challenged enzyme with 25 μL of 80 mM phenylalanine.

4. The variant of claim 1, wherein the substitution increases the residual PAL activity following nebulization relative to the wild-type PAL enzyme.

5. The variant of claim 4, wherein the increased residual PAL activity following nebulization relative to wild-type PAL enzyme is an increase of at least 10% residual activity following a nebulization challenge, wherein residual PAL activity for each enzyme is calculated by dividing the amount of PAL activity following nebulization challenge by the amount of PAL activity prior to nebulization challenge, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

6. The variant of claim 4, wherein the amount of PAL activity is determined by measuring the amount of cinnamic acid produced via liquid chromatography with UV detection following a 30-minute incubation of 21 μg of nebulization challenged enzyme with 25 μL of 80 mM phenylalanine, wherein the nebulization challenge comprises loading 3 mL of liquid enzyme solution with a concentration of 0.5 to 3 g/L into a nebulizer, pulling air into the nebulizer via vacuum to convert the liquid enzyme solution into a mist, capturing the mist from the nebulizer in an ice trap, and recovering a portion of the liquid enzyme sample; and wherein the portion of liquid enzyme sample recovered is between 1 to 2.5 mL.

7. The variant of claim 1, wherein the variant sequence comprises at least one substitution selected from N25E, I27V, N30E, Q31R, N44E, T46V, L47S, I56V, C64V, A70S, Q96E, L104M, T110V, K115L, K115Y, L118K, R134K, S209A, N234L, A246S, M247L, D253H, D282A, 1285L, 1310V, R313K, L316I, Q389N, M416Q, C424T, N437E, A440S, E448D, Q449E, Q457M, S461A, N474V, G483A, C503T, Q521V, and N534D.

8. The variant of claim 1, wherein the variant sequence comprises an amino acid sequence selected from any one of SEQ ID NOs: 2-62.

9. The variant of claim 1, wherein the variant sequence comprises at least two substitutions at two or more amino acid positions selected from 25, 30, 56, 64, 70, 96, 104, 247, 285, 457, 461, 483, 503, and 521 of SEQ ID NO: 1.

10. The variant of claim 1, wherein the variant sequence comprises at least three substitutions selected from N25E, N30E, I56V, C64V, A70S, Q96E, L104M, M247L, I285L, Q457M, S461A, G483A, C503T, and Q521V.

11. The variant of claim 1, wherein the variant sequence comprises at least four substitutions selected from N25E, N30E, C64V, A70S, Q96E, L104M, M247L, I285L, Q457M, S461A, G483A, C503T, and Q521V.

12. The variant of claim 1, wherein the variant sequence comprises at least five substitutions selected from N30E, C64V, A70S, Q96E, L104M, G483A, C503T, and Q521V.

13. The variant of claim 1, wherein the variant sequence comprises at least six substitutions selected from N30E, C64V, A70S, Q96E, L104M, G483A, C503T, and Q521V.

14. The variant of claim 1, wherein the variant sequence comprises at least eight substitutions selected from N30E, C64V, A70S, Q96E, L104M, G483A, C503T, and Q521V.

15. The variants of claim 1, wherein the variant sequence comprises a deletion of a methionine residue at amino acid position 1 of SEQ ID NO: 1.

16. A pharmaceutical composition, comprising: (a) a phenylalanine-degrading enzyme variant according to claim 1, and (b) a pharmaceutically acceptable carrier or excipient, wherein the pharmaceutical composition is formulated for aerosol administration.

17. The pharmaceutical composition of claim 16, wherein the aerosol administration is selected from liquid aerosol administration or dry powder aerosol administration.

18. A pharmaceutical composition, comprising:
(a) a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme; and
(b) a pharmaceutically acceptable carrier.

19. A method of treating a subject with PKU, the method comprising administering to the subject a variant according to claim 1.

20. A method of treating a subject with PKU, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.

21. A method of degrading phenylalanine in a subject, the method comprising administering to the subject a variant according to claim 1.

22. The method of claim 21, wherein the subject has PKU.

23. A method of degrading phenylalanine in a subject, the method comprising administering to the subject a phenylalanine-degrading enzyme variant comprising an amino acid sequence that is a variant sequence of the wild-type phenylalanine ammonia lyase (PAL) enzyme set forth in SEQ ID NO: 1, wherein the variant sequence comprises at least one amino acid substitution relative to SEQ ID NO: 1 that (i) increases thermostability of the enzyme variant relative to the wild-type PAL enzyme, (ii) increases residual PAL activity following nebulization relative to the wild-type PAL enzyme, or (iii) increases thermostability of the enzyme variant relative to the wild-type PAL enzyme and increases residual PAL activity following nebulization relative to the wild-type PAL enzyme.
